SEQUENCE LISTING (14 sequences)

SEQ ID NO: 1 - DNA, 20 bp, Artificial Sequence
  hactin_for
ccaaccgcgagaagatgacc

SEQ ID NO: 2 - DNA, 21 bp, Artificial Sequence
  hactin_rev
gatcttcatgaggtagtcagt

SEQ ID NO: 3 - DNA, 23 bp, Artificial Sequence
  hZEB1_for
gatgatgaatgcgagtcagatgc

SEQ ID NO: 4 - DNA, 19 bp, Artificial Sequence
  hZEB1_rev
ctggtcctcttcaggtgcc

SEQ ID NO: 5 - DNA, 20 bp, Artificial Sequence
  hSLUG_for
gctccttcgtccttctcctc

SEQ ID NO: 6 - DNA, 20 bp, Artificial Sequence
  hSLUG_rev
tgacatctgagtgggtctgg

SEQ ID NO: 7 - DNA, 20 bp, Artificial Sequence
  hSNAIL_for
ggccttcaactgcaaatact

SEQ ID NO: 8 - DNA, 20 bp, Artificial Sequence
  hSNAIL_rev
acatctgagtgggtctggag

SEQ ID NO: 9 - DNA, 24 bp, Artificial Sequence
  hVimentin_for
gacaatgcgtctctggcacgtctt

SEQ ID NO: 10 - DNA, 23 bp, Artificial Sequence
  hVimentin_rev
tcctccgcctcctgcaggttctt

SEQ ID NO: 11 - DNA, 19 bp, Artificial Sequence
  hE-cadh_for
cccaccacgtacaagggtc

SEQ ID NO: 12 - DNA, 19 bp, Artificial Sequence
  hE-cadh_rev
ctggggtattgggggcatc

SEQ ID NO: 13 - DNA, 19 bp, Artificial Sequence
  hN-cadh_for
gccacctacaaaggcagaa

SEQ ID NO: 14 - DNA, 20 bp, Artificial Sequence
  hN-cadh_rev
atgtgccctcaaatgaaacc

(19) United States
(12) Patent Application Publication
(10) Pub. No.: US 2023/0137849 A1
(43) Pub. Date: May 4, 2023

(54) USE OF A COMPOUND OF THE DIURETICS CLASS FOR TREATING CANCER

(71) Applicant: AZELEAD, MONTPELLIER CEDEX 5 (FR)

(72) Inventors: Laura FONTENILLE, MONTFERRIER SUR LEZ (FR); Amel KISSA, CLICHY (FR); Karima KISSA, MONTPELLIER (FR)

(21) Appl. No.: 16/484,877

(22) PCT Filed: Feb. 9, 2018
(86) PCT No.: PCT/FR2018/000020

§ 371 Date: Aug. 9, 2019

(30) Foreign Application Priority Data
Feb. 10, 2017 (FR) 17/51118

(51) Int. Cl. A61K 31/549; A61P 35/04; A61K 31/704; A61K 45/06

(52) U.S. Cl. CPC A61K 31/549; A61P 35/04; A61K 31/704; A61K 45/06

(57) ABSTRACT

Disclosed is a cancer treatment method utilizing a compound of the diuretics class. Also disclosed is a pharmaceutical composition used in such cancer treatment method, the compound being althiazide.

### SEQUENCE LISTING

An attached Sequence Listing (i. Name: SEQCRF_0508-1456_ST25, ii. Date of Creation: Oct. 15, 2019, and iii. Size: 2,846 bytes) is based on the Sequence Listing filed with U.S. application Ser. No. 16/484,877.

The invention relates to the use of a compound of the diuretics class for treating cancer.

The treatment of cancer can be done at different stages of the disease, in particular by treating primary cancer tumors or preventing the occurrence and/or treatment of secondary cancerous tumors. The occurrence of cancer metastases is the leading cause of death in cancer patients. The identification of new drugs capable of preventing the metastatic progression of a cancerous tumor is therefore essential.

Dasatinib is a potent inhibitor of many tyrosine kinases (mainly BCR-Abl and Src), which act by lodging in the active site of the kinase in competition with ATP. The deregulated action of these tyrosine kinases leads to an abnormal proliferation of myeloid cells causing the appearance of leukemias. Dasatinib is primarily used for the treatment of chronic myeloid leukemia resistant to Imatinib.

Anti-metastatic effects of dasatinib have also been observed in various experimental models, but this drug has not been selected as an antimetastatic treatment in humans, mainly because of its low tolerability by patients (fatigue, hyponatremia, diarrhea, gastrointestinal bleeding, pleural and pericardial effusions and anemia) which can be explained by its inhibitory activity of tyrosine kinases.

Althiazide (CAS number: 5588-16-9) is a thiazide diuretic used primarily in combination with spironolactone, which acts at the nephron level by inhibiting sodium reabsorption, resulting in increased diuresis and sodium and chlorides excretion. The state of the art puts forward a correlation between hypertension, antihypertensive treatments and cancer-related mortality. If the literature seems to show an increase in cancer-related mortality among patients with hypertension, it would also appear that there is an incidence of cancer following treatment for hypertension. Indeed, diuretics, which are mainly thiazides, could be a risk factor for renal cancer. On this point, the published results are very contradictory, and do not allow to conclude if the correlation is direct or indirect and if it can be extended to all the cancers or if it is restricted to the renal cancers.

Another study analyzes the impact of hypertension and antihypertensive treatments on cancer mortality in women. The observed results are variable and differ according to the types of cancers. For patients taking antihypertensive treatment, the mortality associated with certain cancers seems to decrease, whereas it is increased for others.

There is therefore a lack of tolerable therapeutic molecules for patients and effective for the treatment or prevention of diseases caused or exacerbated by the proliferation and dispersion of cancerous tumor cells.

This is why one of the aims of the invention is to provide a compound for its use in the treatment of cancer.

Another object of the invention is to provide a compound for use in treating primary and secondary cancer tumors and/or preventing the occurrence and development of secondary cancerous tumors.

The present invention relates to a family of compounds for use in the treatment of cancer, said family of compounds being of formula (II)

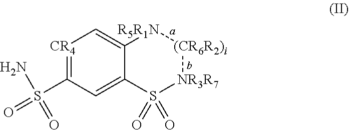

in which:
a can represent:
nothing,
a single bond,
a double bond;
b represents:
nothing if a represents nothing,
a single bond if a is either a single bond or a double bond;
the value of i being:
zero if a represents nothing,
equal to 1 if a is a single or a double bond;
if a represents nothing, then b represents nothing and i is 0:
R₁ and R₅ can represent independently of one another:
H;
a linear or branched alkyl or alkenyl chain of 1 to 20 carbon atoms, monocyclic or polycyclic of 3 to 20 carbon atoms, in particular bicyclic, optionally containing one or more heteroatoms chosen from N, O, S, SO, SO₂, and being optionally substituted with 1 or more halogen atoms;
an aryl group of 1 to 20 carbons, optionally substituted with one or more alkyl chains and optionally comprising 1 or more heteroatoms or functions selected from N, O, S, SO, SO₂, and/or 1 or more halogen atoms;
R₃ and R₇ can represent independently of one another:
H;
a linear or branched alkyl or alkenyl chain of 1 to 20 carbon atoms, monocyclic or polycyclic of 3 to 20 carbon atoms, in particular bicyclic, optionally containing one or more heteroatoms chosen from N, O, S, SO, SO₂, and being optionally substituted with 1 or more halogen atoms;
an aryl group of 1 to 20 carbons, optionally substituted with one or more alkyl chains and optionally comprising 1 or more heteroatoms or functions selected from N, O, S, SO, SO₂, and/or 1 or more halogen atoms;
R₄ can represent:
H;
a halogen atom
a linear or branched alkyl or alkenyl chain of 1 to 20 carbon atoms, monocyclic or polycyclic of 3 to 20 carbon atoms, in particular bicyclic, optionally containing one or more heteroatoms chosen from N, O, S, SO, SO₂, and being optionally substituted with 1 or more halogen atoms;
if a represents a single bond, then b represents a single bond and i is equal to 1:
R₁ can represent:
H;
a linear or branched alkyl or alkenyl chain of 1 to 20 carbon atoms, monocyclic or polycyclic of 3 to 20 carbon atoms, in particular bicyclic, optionally containing one or more heteroatoms chosen from N, O, S, SO, SO₂, and being optionally substituted with 1 or more halogen atoms;
an aryl group of 1 to 20 carbons, optionally substituted with one or more alkyl chains and optionally comprising 1 or more heteroatoms or functions selected from N, O, S, SO, SO₂, and/or 1 or more halogen atoms;
R₂ can represent:
H;
a linear or branched alkyl or alkenyl chain of 1 to 20 carbon atoms, monocyclic or polycyclic of 3 to 20 carbon atoms, in particular bicyclic, optionally containing one or more heteroatoms chosen from N, O, S, SO, SO₂, and being optionally substituted by 1 or more halogen atoms, said heteroatoms optionally forming functions, in particular functions selected from NRₐR_{b}, ORₐ, SRₐ, SORₐ, SO₂Rₐ, SO₂NRₐR_{b}, CONRₐR_{b}, in which Rₐ and/or R_{b} represent, independently, a hydrogen, a linear, branched or cyclic alkyl or alkenyl chain of 1 to 20 carbons, optionally with heteroatoms and/or halogens, or an aromatic ring of 1 to 20 carbons, optionally containing hetero atoms and being optionally substituted;
an aryl group of 1 to 20 carbons, optionally substituted with one or more alkyl chains and optionally comprising 1 or more heteroatoms or functions selected from N, O, S, SO, SO₂, and/or 1 or more halogen atoms, said heteroatoms optionally forming functions, in particular functions chosen from NRₐR_{b}, ORₐ, SRₐ, SORₐ, SO₂Rₐ, SO₂NRₐR_{b}, CONRₐR_{b}, in which Rₐ and/or R_{b} represent, independently, a hydrogen, a linear, branched or cyclic alkyl or alkenyl chain of 1 to 20 carbons, optionally with heteroatoms and/or halogens, or an aromatic ring of 1 to 20 carbons, optionally containing hetero atoms and being optionally substituted;
R₃ can represent:
H;
a linear or branched alkyl or alkenyl chain of 1 to 20 carbon atoms, monocyclic or polycyclic of 3 to 20 carbon atoms, in particular bicyclic, optionally containing one or more heteroatoms chosen from N, O, S, SO, SO₂, and being optionally substituted with 1 or more halogen atoms;
an aryl group of 1 to 20 carbons, optionally substituted with one or more alkyl chains and optionally comprising 1 or more heteroatoms or functions selected from N, O, S, SO, SO₂, and/or 1 or more halogen atoms;
R₅ and R₇ represent nothing
R₆ represents H
R₄ is as defined above
if a represents a double bond, then b represents a single bond and i is 1:
R₁, R₅, R₆ and R₇ represent nothing;
R₂, R₃ and R₄ are as defined above.

For the purposes of the present invention, “cancer treatment” refers both to the treatment of the primary cancer tumor by preventing the local progression thereof and to the prevention of formation of secondary cancerous tumors also called cancerous metastases, in distant tissues.

A “cancerous tumor” is defined by the development of a newly formed tissue within a normal tissue. The cancerous tumor is caused by the dysfunction of cellular development.

The present invention also relates to a family of compounds for use in the treatment of cancer, said family of compounds being of formula (III)

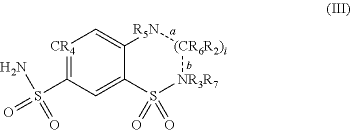

in which:

a can represent:
nothing;
a single bond;
a double bond.
b represents:
nothing if a=nothing
a single bond if a is either a single bond or a double bond
the value of i being:
zero if a represents nothing,
equal to 1 if a is a single or a double bond;
if a represents nothing, then b represents nothing and i is 0:
R₃ and R₇ can represent independently of one another:
H;
a linear or branched alkyl or alkenyl chain of 1 to 20 carbon atoms, monocyclic or polycyclic of 3 to 20 carbon atoms, in particular bicyclic, optionally containing one or more heteroatoms chosen from N, O, S, SO, SO₂, and being optionally substituted with 1 or more halogen atoms;
R₄ can represent:
H;
a halogen atom
a linear or branched alkyl or alkenyl chain of 1 to 20 carbon atoms, monocyclic or polycyclic of 3 to 20 carbon atoms, in particular bicyclic, optionally containing one or more heteroatoms chosen from N, O, S, SO, SO₂, and being optionally substituted with 1 or more halogen atoms;
R₅ represents H₂;
if a represents a single bond, then b represents a single bond and i is 1:
R₂ can represent:
H,
a linear or branched alkyl or alkenyl chain of 1 to 20 carbon atoms, monocyclic or polycyclic of 3 to 20 carbon atoms, in particular bicyclic, optionally containing one or more heteroatoms chosen from N, O, S, SO, SO₂, and being optionally substituted by 1 or more halogen atoms, said heteroatoms optionally forming functions, in particular functions selected from SRₐ, SORₐ, SO₂Rₐ, SO₂NRₐR_{b}, in which Rₐ and/or R_{b} represent, independently hydrogen, an alkyl chain or linear, branched or cyclic alkenyl of 1 to 20 carbons, optionally with heteroatoms and/or halogens, or an aromatic ring of 1 to 20 carbons, optionally containing hetero atoms and being optionally substituted;
an aryl group of 1 to 20 carbons, optionally substituted with one or more alkyl chains and optionally comprising 1 or more heteroatoms or functions selected from N, O, S, SO, SO₂, and/or 1 or more halogen atoms, said heteroatoms optionally forming functions, in particular functions selected from SRₐ, SORₐ, SO₂Rₐ, SO₂NRₐR_{b}, in which Rₐ and/or R_{b} represent, independently hydrogen, a linear, branched or cyclic alkyl or alkenyl chain of 1 to 20 carbons, with optionally heteroatoms and/or halogens, or an aromatic ring of 1 to 20 carbons, optionally containing hetero atoms and being optionally substituted;
R₃ can represent:
H;
a linear or branched alkyl or alkenyl chain of 1 to 20 carbon atoms, monocyclic or polycyclic of 3 to 20 carbon atoms, in particular bicyclic, optionally containing one or more heteroatoms chosen from N, O, S, SO, SO₂, and being optionally substituted with 1 or more halogen atoms;
R₅ and R₆ represent H;
R₇ represent nothing;
R₄ is as defined above
if a represents a double bond, then b represents a single bond and i is equal to 1:
R₅, R₆ and R₇ represent nothing
R₂, R₃ and R₄ are as defined above

The present invention also relates to a family of compounds for use in the treatment of cancer, said family of compounds being of formula (III)

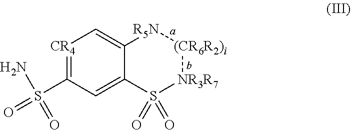

in which:
a can represent:
nothing;
a single bond;
a double bond,
b represents:
nothing if a represents nothing;
a single bond if a is either a single bond or a double bond
the value of i being:
zero if a represents nothing;
equal to 1 if a is a single or a double bond;
if a represents nothing, then b represents nothing and i is 0:
R₃ and R₇ can represent independently of one another:
H;
a linear alkyl chain of 1 carbon atom, optionally substituted with 1 or more halogen atoms;
R₅ represents H₂;
R₄ can represent:
H;
a halogen atom
a linear alkyl chain of 1 carbon atom, optionally substituted with 1 or more halogen atoms;
if a represents a single bond, then b represents a single bond and i is equal to 1:
R₂ can represent:
H;
a linear or branched alkyl or alkenyl chain of 1 to 10 carbon atoms, monocyclic or polycyclic of 3 to 20 carbon atoms, in particular bicyclic, containing. optionally one or more heteroatoms selected from S and SO₂, and being optionally substituted with 1 or more halogen atoms, said heteroatoms possibly forming functions, in particular functions chosen from SRₐ, and SO₂NRₐR_{b}, in which Rₐ and/or R_{b} represent, independently, a hydrogen or a linear alkyl or alkenyl chain of 1 to 10 carbons;
an aryl group of 1 to 20 carbons, optionally substituted by one or more alkyl chains and optionally comprising 1 or more heteroatoms or functions chosen from, S and SO₂, and/or 1 or more halogen atoms, said heteroatoms possibly forming functions, in particular functions selected from SRₐ, SO₂NRₐR_{b}, in which Rₐ and/or R_{b} represent, independently hydrogen or a linear alkyl or alkenyl chain of 1 to 10 carbons;
R₃ can represent:
H;
a linear alkyl chain of 1 carbon atom, optionally substituted with 1 or more halogen atoms;
R₅ and R₆ represent H;
R₇ represents nothing;
R₄ is as defined above
if a represents a double bond, then b represents a single bond and i is equal to 1:
R₅, R₆ and R₇ represent nothing
R₂, R₃ and R₄ are as defined above

The present invention also relates to a family of compounds for use in the treatment of cancer, said family of compounds being of formula (IV)

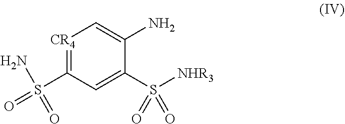

in which:
R₃ can represent:
H;
a linear alkyl chain of 1 carbon atom, optionally substituted with 1 or more halogen atoms;
R₄ can represent:
H;
a halogen atom
a linear alkyl chain of 1 carbon atom, optionally substituted with 1 or more halogen atoms;

The present invention also relates to a family of compounds for use in the treatment of cancer, said family of compounds being of formula (V):

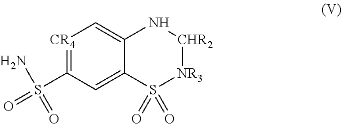

in which:
R₂ can represent:
H;
a linear or branched alkyl or alkenyl chain of 1 to 10 carbon atoms, monocyclic or polycyclic of 3 to 20 carbon atoms, in particular bicyclic, optionally containing one or more heteroatoms chosen from S and SO₂, and being optionally substituted with 1 or a plurality of halogen atoms, said heteroatoms optionally forming functions, in particular functions selected from SRₐ, SO₂NRₐR_{b}, in which Rₐ and/or R_{b} represent, independently hydrogen or a linear alkyl or alkenyl chain of 1 to 10 carbons;
an aryl group of 1 to 20 carbons, optionally substituted by one or more alkyl chains and optionally comprising 1 or more heteroatoms or functions chosen from, S and SO₂, and/or 1 or more halogen atoms, said heteroatoms possibly forming functions, in particular functions selected from SRₐ, and SO₂NRₐR_{b} wherein Rₐ and/or R_{b} are, independently, hydrogen or a linear alkyl or alkenyl chain of 1 to 10 carbons;
R₃ can represent:
H;
a linear alkyl chain of 1 carbon atom, optionally substituted with 1 or more halogen atoms;
R₄ can represent:
H;
a halogen atom
a linear alkyl chain of 1 carbon atom, optionally substituted with 1 or more halogen atoms;

The present invention also relates to a family of compounds for use in the treatment of cancer, said family of compounds being of formula (VI):

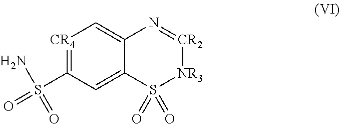

in which:
R₂ can represent:
H;
a linear or branched alkyl or alkenyl chain of 1 to 10 carbon atoms, optionally comprising one or more heteroatoms chosen from S and SO₂, and being optionally substituted with 1 or more halogen atoms, said heteroatoms possibly forming functions, in particular functions selected from SRₐ, SO₂NRₐR_{b}, in which Rₐ and/or R_{b} represent, independently, hydrogen or a linear alkyl or alkenyl chain of 1 to 10 carbons;
an aryl group of 1 to 20 carbons, optionally substituted by one or more alkyl chains and optionally comprising 1 or more heteroatoms or functions chosen from, S and SO₂, and/or 1 or more halogen atoms, said heteroatoms possibly forming functions, in particular functions selected from SRₐ, SO₂NRₐR_{b}, in which Rₐ and/or R_{b} represent, independently hydrogen or a linear alkyl or alkenyl chain of 1 to 10 carbons;
R₃ can represent:
H;
A linear alkyl chain of 1 carbon atom, optionally substituted with 1 or more halogen atoms;
R₄ can represent:
H;
a halogen atom
A linear alkyl chain of 1 carbon atom, optionally substituted with 1 or more halogen atoms.

The present invention also relates to a compound for its use in the treatment of cancer; said compound being althiazide of formula (I)

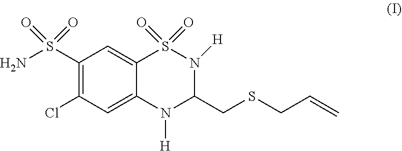 [SMILES: C=CCSCC1Nc2cc(Cl)c(S(N)(=O)=O)cc2S(=O)(=O)N1]

The althiazide of formula (I) comprises the set of form of althiazide corresponding to this formula, namely:
The enantiomer of formula (IA)

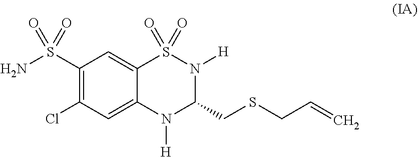 [SMILES: C=CCSC[C@@H]1Nc2cc(Cl)c(S(N)(=O)=O)cc2S(=O)(=O)N1]

The enantiomer of formula (IB)

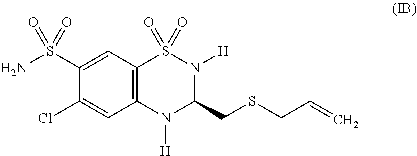 [SMILES: C=CCSC[C@H]1Nc2cc(Cl)c(S(N)(=O)=O)cc2S(=O)(=O)N1]

The racemic form,
All the mixtures of the forms (IA) and (IB), with contents of enantiomer (IA) ranging from 1% to 99%, in particular ranging from 0% to 10%, from 10% to 20%, from 20% to 30%, from 30% to 40%, from 40% to 50%, from 50% to 60%, from 60% to 70%, from 70% to 80%; from 80% to 90% and from 90% to 100%, said contents being measured in particular according to the method of analysis of the chiral purity described in the Materials and Methods section.

The present invention thus relates to a compound for use in the treatment of cancer; said compound being the althiazide of formula (IA),

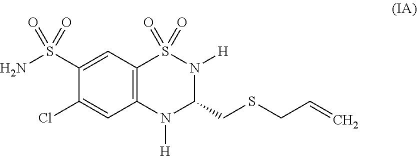 [SMILES: C=CCSC[C@@H]1Nc2cc(Cl)c(S(N)(=O)=O)cc2S(=O)(=O)N1]

and being in pure form or with a chiral purity greater than 99%

The chiral purity greater than 99% is measured for example according to the method of analysis of the chiral purity described in the Materials and Methods section. The present invention thus relates to a compound for use in the treatment of cancer; said compound being the althiazide of formula (IB),

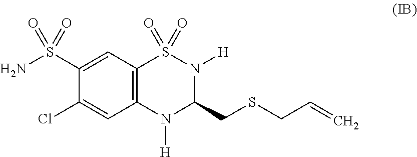 [SMILES: C=CCSC[C@H]1Nc2cc(Cl)c(S(N)(=O)=O)cc2S(=O)(=O)N1]

and being in pure form or with a chiral purity greater than 99%.

The chiral purity greater than 99% is measured for example according to the method of analysis of the chiral purity described in the Materials and Methods section.

The present invention also relates to a compound for its use in the treatment of cancer; said compound being the racemic form of althiazide.

Within the meaning of the present invention, the term “racemic form of althiazide” is understood to mean an enantiomer mixture of althiazide in which the respective chiral purities of the enantiomers of formula (IA) and (IB) are comprised from 49.5% to 50.5%, measured in particular according to the method of analysis of the chiral purity described in the Materials and Methods section.

The present invention also relates to a compound for its use in the treatment of cancer; said compound being of formula:

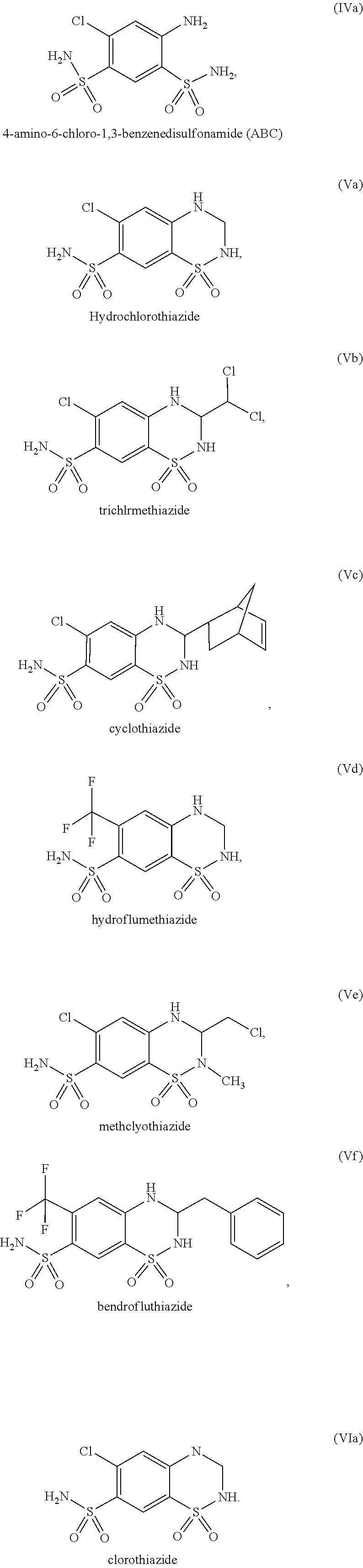

The present invention also relates to a compound for its use in the treatment of cancer, said family of compounds being of formula (VIIa) or (VIIIb):

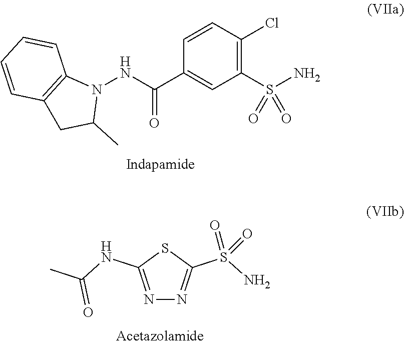 [SMILES: CC1Cc2ccccc2N1NC(=O)c1ccc(Cl)c(S(N)(=O)=O)c1 ; CC(=O)Nc1nnc(S(N)(=O)=O)s1]

According to one embodiment, the present invention relates to the compound according to the invention of formula (II) for its use in combination with an anticancer compound in the treatment of primary cancer tumors.

For the purposes of the present invention, the term “primary cancer tumor” means a tumor arising at the level of an organ.

The present invention also relates to the thiazide of formula (I) or an enantiomer of formula (IA) or (TB) for its use in combination with an anticancer compound as soon as the primary tumor appears in order to prevent the progression of this latter locally.

The present invention also relates to a compound of formula (IVa), (Va), (Vb), (Vc), (Vd), (Ve), (Vf), (VIa), (VIIa) or (VIIb) for its use in combination with an anticancer compound from the onset of the primary tumor to prevent the progression of it locally.

According to another embodiment, the present invention relates to the compound of formula (II) according to the invention for use in combination with an anticancer compound in the treatment of cancer in the pre-metastatic state.

The present invention also relates to the althiazide of formula (I) or an enantiomer of formula (IA) or (IB) for its use in combination with an anticancer compound in the treatment of cancer in the pre-metastatic state.

The present invention also relates to a compound of formula (IVa), (Va), (Vb), (Vc), (Vd), (Ve), (Vf), (VIa), (Vlla) or (Vllb) for its use in combination with an anticancer compound in the treatment of cancer in the pre-metastatic state.

Althiazide, used alone, has an anti-metastatic effect under normal physiological conditions. For the purposes of the invention, the “treatment of cancer in the pre-metastatic state” refers to the prevention of secondary cancerous tumors. It is a treatment of cancer before the appearance of metastases, in the case of tumors considered “high risk” of metastases or from the appearance of the first metastases and the prevention of the appearance of the following. The terms “secondary cancerous tumor” or “metastasis” define the abnormal development of tissues from migration through the bloodstream or lymphatic circulation of cancer cells. For the purpose of the present invention, the term “high-risk” tumors of metastases” refers to the last stage of stratification of patients, determined according to the stage of development of the tumor, the Gleason score and the value of PSA. For example, high-risk prostate cancers are locally advanced (T2c and T3 clinical), high-grade (Gleason 4+3=7 and greater than 7) or associated with a total PSA value>20 ng/ml or whose kinetics is >2 ng/ml/year or the doubling time <3 months.

For the purposes of the present invention, an anti-migratory effect is therefore considered to be a good indicator in vivo of the anti-metastatic activity.

A method for determining whether a molecule is capable of treating cancer in the pre-metastatic state has been described in international application WO 2011/007259. This method consists in isolating molecules capable of inhibiting the emergence of hematopoietic stem cells from the floor of the aorta without cell division, but by endothelial-to-hematopoietic transition. This emergence involves the loss of cell polarity of emerging cells, the loss of cellular junctions with these neighboring cells and the acquisition of migratory properties. This model is used because it mimics each stage of the epithelial-mesenchymal transition, the first stage of cancer metastasis. The endothelial-to-hematopoietic transition takes place in a region of the embryo called Aorta-Gonad-Mesonephros (AGM).

The colonization of hematopoietic tissues by hematopoietic stem cells mimics the appearance and/or progression of a metastatic cancerous tumor since it involves intravasation, extravasation, or the migration of HSCs following a gradient of SDF-1. The first colonized tissue is caudal hematopoietic tissue (CHT).

In order to evaluate the inhibition of the endothelial-to-hematopoietic transition, transgenic CD41:GFP (green fluorescent protein) embryos are used. HSCs express GFP under the control of the CD41 promoter, which allows them to be monitored in vivo under a fluorescence microscope. The read-out is the number of hematopoietic stem cells CD41:GFP accumulated in the AGM (mimicking the initiation of the metastatic process) and/or in the CHT of the zebrafish embryo (mimicking the progression of the metastatic process). The accumulation of CD41:GFP cells in CHT implies that each cell has made an endothelial-to-hematopoietic transition, intravasation, extravasation at the CHT level, has settled in a stromal niche and has proliferated. At the same time, the phenotypic characterization of embryos makes it possible to isolate compounds capable of preventing the endothelial-to-hematopoietic transition and their migration to CHT without causing toxic effects on the developing embryo.

According to another embodiment, the present invention relates to the compound of formula (II) according to the invention for its use in the treatment of primary and secondary cancer tumors.

The present invention also relates to the compound of formula (I), (IA) or (IB) according to the invention for its use in the treatment of primary and secondary cancer tumors.

The present invention also relates to the compound of formula (IVa), (Va), (Vb), (Vc), (Vd), (Ve), (Vf), (VIa), (VIIa) or (VIIb) according to invention for its use in the treatment of primary and secondary cancer tumors.

The compound of the invention thus relates to use in patients who may be suffering from cancer at different stages of the disease: for the treatment of primary cancer tumors only or after migration and presence of metastases.

Under conditions of hypoxia, that is to say under the conditions found within a cancerous tumor, the althiazide of formula (I) is cytotoxic.

For the purposes of the invention, the “hypoxia conditions” correspond to a decrease in the oxygen content of from 1% to 0.1%. This condition is found in rapidly growing tumors for which the microcirculation within the cells is diminished.

According to one embodiment, the present invention relates to the compound according to the invention of formula (II) for its use as an anti-cancer agent or potentiator of an anti-cancer agent, in particular as an anti-metastatic agent, in the treatment of cancer.

The present invention also relates to the compound according to the invention of formula (I), (IA) or (IB) for its use as an anti-cancer agent or potentiator of an anti-cancer agent, in particular as an anti-metastatic agent, in the treatment of cancer.

The present invention also relates to the compound according to the invention of formula (IVa), (Va), (Vb), (Vc), (Vd), (Ve), (Vf), (VIa), (VIIa) or (VIIb) for its use as an anti-cancer agent or potentiator of an anti-cancer agent, in particular as an anti-metastatic agent, in the treatment of cancer.

For the purposes of the present invention, the term “anti-cancer agents” denotes a compound that makes it possible to fight against cancer.

For the purposes of the invention, the expression “potentiator of an anti-cancer agent” designates a compound capable of improving the anti-cancer properties of an anti-cancer agent.

According to another embodiment, the present invention relates to the compound according to the invention for its use as an anti-cancer agent in the treatment of cancer; said compound being althiazide of formula (I)

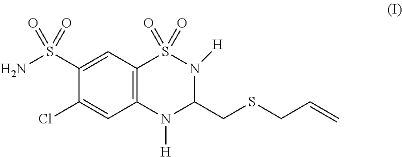 [SMILES: C=CCSCC1Nc2cc(Cl)c(S(N)(=O)=O)cc2S(=O)(=O)N1]

According to another embodiment, the present invention relates to the compound of formula (I) according to the invention for its use as anti-metastatic agent in the treatment of cancer, alone or in combination with:
an anti-mitotic agent, in particular vinca-alkaloid, dolastatin, taxane or epothilone,
an anti-metabolite agent, in particular a pyrimidine analogue, a purine analogue or a folic acid analogue),
an alkylating agent, in particular nitrogen mustard, oxazaphosphorine, triazene and hydrazine, ethylene imine, nitrosourea, alkyl or alkane sulfonate or organoplatin,
a DNA modifying agent, in particular a topoisomerase I and II inhibitor,
an anti-angiogenic agent, in particular a molecule interacting with the tumor angiogenesis activation pathways, a directly anti-angiogenic molecule, or an inhibitor of the matrix metalloproteinases,
a monoclonal antibody.

According to another embodiment, the present invention relates to the compound according to the invention for its use as potentiator of an anti-cancer agent in the treatment of cancer; said compound being althiazide of formula (I)

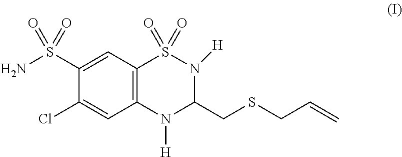 [SMILES: C=CCSCC1Nc2cc(Cl)c(S(N)(=O)=O)cc2S(=O)(=O)N1]

When administered with other anti-cancer molecules active under normal physiological conditions, althiazide is cytotoxic. When administered with other active anti-cancer molecules under hypoxia conditions, althiazide is cytotoxic.

For the purposes of the invention, “normal physiological conditions” are defined by conditions in which the oxygen content is 21%. This condition is also called normoxia, as opposed to hypoxia conditions in which the oxygen level is greatly decreased (from 1% to 0.1%).

The althiazide of formula (I) potentiates the anticancer effects of other drugs. The inventors have thus observed an improvement in the drugs already used as anticancer drugs after the addition of althiazide. For example, rapamycin is no longer active under hypoxic tumor conditions and althiazide restores this activity.

A cytotoxicity test (MTT) can show a restoration or improvement of the cytotoxic activity of anti-cancer molecules in combination with althiazide. The principle of this test is based on the reduction of the tetrazolium to formazan cycle by the mitochondrial succinate dehydrogenase of active living cells, leading to the formation of a violet color precipitate in the mitochondria. The amount of precipitate formed is proportional to the amount of living cells (but also to the metabolic activity of each cell). After incubation of the cells with MTT for 3 h at 37° C., the cells, their mitochondria and thus the purple formazan precipitates are dissolved in a DMSO/EtOH mixture (1:1). A spectroscopic assay of the optical density at a wavelength of 570 to 590 nm makes it possible to know the relative quantity of living and metabolically active cells. The test is the same in normal condition and in hypoxia condition.

Althiazide potentiates the cytotoxic effect of treatments administered to patients at different stages of cancer, both on primary cancer tumors and secondary cancer tumors.

Althiazide has the effect of potentiating and/or reducing the doses of other anticancer agents and thus reducing the problems of tolerability.

According to one embodiment, the present invention relates to the compound of formula (I) according to the invention for use in combination with another anti-cancer agent in the treatment of cancer.

According to one embodiment, the present invention relates to the compound of formula (I) according to the invention for its use as anti-cancer agent or potentiator of an anti-cancer agent, said other anti-cancer agent being a radiation or a chemical agent, in particular a chemotherapy agent, a radiotherapy agent, a radio-pharmaceutical or an anti-angiogenic agent.

According to one embodiment, the present invention relates to the compound of formula (I) for its use according to the invention, said other anti-cancer agent being chosen from
alkylating agents such as alkylsulfonates in particular busulfan, dacarbazine, procarbazine, cloretazine, nitrogen mustards such as chlormethine, melphalan, chlorambucil, cyclophosphamide, ifosfamide, nitrosoureas such as carmustine, lomustine, semustine, streptozocin, altretamine, fotemustine;
antineoplastic alkaloids such as vincristine, vinblastine, vinorelbine, vindesine;
taxanes such as paclitaxel or taxotere;
antineoplastic antibiotics such as actinomycin, bleomycin;
intercalating agents such as mitoxantrone, etoposide, bleomycin, actinomycin D, amsacrine, alliptinium;
antineoplastic antimetabolites: folate antagonists, methotrexate; inhibitors of purine synthesis; purine analogues such as mercaptopurine, 6-thioguanine; inhibitors of pyrimidine synthesis, aromatase inhibitors, capecitabine, pyrimidine analogs such as fluorouracil, gemcitabine, cytarabine and cytosine arabinoside; brequinar, nelarabine;
group I and II topoisomerase inhibitors such as irinotecan, exatecan, topotecan, teniposide, camptothecin or etoposide;
anticancer hormone agonists and antagonists including tamoxifen;
kinase inhibitors, such as imatinib, nilotinib and dasatinib, midaustorin, sorafenib, lestaurtinib, tandutinib, sirolimus, everolimus or tensirolimus;
growth factor inhibitors;
anti-inflammatories such as pentosan polysulfate, corticosteroids, prednisone, dexamethasone;
ceplene (histamine dihydrochloride);
antracyclines such as daunorubicin, epirubicin, pirarubicin, idarubicin, zorubicin, aclarubicin, annamycin, doxorubicin, mitomycin and methramycin;
anticancer metal complexes, platinum derivatives such as cisplatin, carboplatin, oxaliplatin, satraplatin;
alpha interferon;
triphenylthiophosphoramide;
antiangiogenic agents;
thalidomide;
inhibitors of farnesyl-tranferase such as tipifarnib;
inhibitors of DNA methyltransferase such as MG98;
immunotherapy adjuvants such as gemtuzumab ozogamicin, HuM 195;
biotherapeutic agents such as CT388-I L3;
antisense such as GTI-2040;
vaccines.

According to one embodiment, the present invention relates to the compound of formula (II) for its use according to the invention, said compound being formulated to be administered to humans or animals at a dosage from 0.016 mg/kg to 16 mg/kg in admixture with pharmaceutically acceptable excipients. The present invention also relates to the compound of formula (I), (IA) or (IB) for its use according to the invention, said compound being formulated to be administered to humans or animals at a dosage from 0.016 mg/kg to 16 mg/kg, in admixture with pharmaceutically acceptable excipients.

The present invention also relates to the compound of formula (IVa), (Va), (Vb), (Vc), (Vd), (Ve), (Vf), (VIa), (VIIa) or (VIIb) for its use according to the invention, said compound being formulated to be administered to humans or animals at a dosage from 0.016 mg/kg to 16 mg/kg, in admixture with pharmaceutically acceptable excipients.

Within the meaning of the present invention, it is understood by “pharmaceutically acceptable” what is useful in the preparation of a pharmaceutical composition which is of purity and quality sufficient for veterinary as well as human pharmaceutical use.

“Pharmaceutically acceptable excipients” are substances which are non-toxic, biologically tolerable and biologically capable of being administered to a subject, such as an inert substance, added to a pharmacological composition or used as a vehicle, carrier or diluent to facilitate the administration of an agent and who is compatible with it. Examples of pharmaceutically acceptable excipients include, for example, within the meaning of the invention, oils, surfactants (such as Tween), alcohols, polyols, glycerol and vegetable oils, water, solutions salines, glycerol solutions, ethanol, N-(1-(2,3-dioleyloxy)propyl)-N,N,N-trimethylammonium (DOTMA), diolesylphosphatidylethanolamine (DOPE), and liposomes.

According to another embodiment, the present invention relates to the compound of formula (I) for its use according to the invention, said compound being formulated to be administered to humans at a dosage from 0.016 mg/kg to 1.6 mg/kg in admixture with pharmaceutically acceptable excipients. Below 0.016 mg/kg of active substance, the althiazide of formula (I) is not effective when administered to humans. Above 1.6 mg/kg of active substance, the althiazide of formula (I) causes toxicity in humans.

According to another embodiment, the present invention relates to the compound of formula (I) for its use according to the invention, said compound being formulated to be administered to animals at a dosage from 0.1 mg/kg to 16 mg/kg in admixture with pharmaceutically acceptable excipients.

For the purposes of the invention, an administration to animals includes an administration, in particular to domestic animals such as dogs or cats.

Below 0.1 mg/kg of active substance, the althiazide of formula (I) is not effective during administration to the animal. Above 16 mg/kg of active substance, the althiazide of formula (I) causes toxicity in the animal.

According to one embodiment, the present invention relates to the compound of formula (I) for its use according to the invention, said compound being formulated to be administered, to humans or animals, in unitary form from 1 mg to 160 mg, in admixture with pharmaceutically acceptable excipients.

According to another embodiment, the present invention relates to the compound of formula (I) for its use according to the invention, said compound being formulated to be administered to humans in unit form from 1 mg to 100 mg, in admixture with pharmaceutically acceptable excipients. Below 1 mg of active substance, the althiazide of formula (I) is not effective when administered to humans. Above 100 mg of active substance, the althiazide of formula (I) causes toxicity in humans.

According to another embodiment, the present invention relates to the compound of formula (I) for its use according to the invention, said compound being formulated to be administered to animals in unit form from 1 mg to 160 mg, mixed with excipients pharmaceutically acceptable. Below 1 mg of active substance, the althiazide of formula (I) is not effective when administered to the animal. Above 160 mg of active substance, the althiazide of formula (I) causes toxicity in animals.

According to another embodiment, the present invention relates to a method for the treatment of cancer comprising the administration, to humans, of the compound of formula (I) according to the invention, used as an anti-cancer agent or potentiator of an anticancer agent, at a dosage from 1 mg/kg/day to 100 mg/kg/day, in admixture with pharmaceutically acceptable excipients.

According to one embodiment, the present invention relates to a method for the treatment of cancer comprising administering to animals the compound of formula (I) according to the invention, used as anti-cancer agent or potentiator of an anti-cancer agent. at a dosage of 1 mg/kg/day to 160 mg/kg/day, in admixture with pharmaceutically acceptable excipients.

When the althiazide of formula (I), according to the invention, is used as potentiator of an anti-cancer agent, the doses of active substances as defined above are lower, because of a synergy between the althiazide of formula (I) and the anti-cancer agent in combination

According to another embodiment, the present invention relates to a method for the treatment of cancer comprising administering to animals the compound of formula (I) according to the invention, at a dosage from 1 mg/kg/day to 160 mg/kg/day, in admixture with pharmaceutically acceptable excipients.

When the althiazide of formula (I), according to the invention, is used as potentiator of an anti-cancer agent, the doses of active substances as defined above are lower, because of a synergy between the althiazide of formula (I) and the anti-cancer agent in combination.

According to one embodiment, the present invention relates to the compound of formula (I) for its use as anti-cancer agent or potentiator of an anti-cancer agent according to the invention, said compound being used by oral administration, parenteral , inhalation spray, nasal, vaginal, rectal, sub-lingual or local, in particular topical.

By “parenteral routes” is meant, for example, the intramuscular, intraperitoneal, intravenous, intracerebroventricular, intracisternal or subcutaneous routes. Preferably the compound of the invention is used by administration “orally”. Oral administration is preferred for an anti-metastatic compound that is given as daily and long-term (several years) treatment.

Suitable oral dosage unit forms include tablets, soft or hard capsules, capsules, powders, granules, oral solutions or suspensions, and intravenous forms of administration.

When preparing a solid composition in tablet form, the main active ingredient is mixed with a pharmaceutical carrier such as gelatin, starch, lactose, magnesium stearate, talc, gum arabic or the like. The tablets can be coated with sucrose or other suitable materials or they can be treated in such a way that they have prolonged or delayed activity and continuously release a predetermined amount of active ingredient.

A preparation in capsules is obtained by mixing the active ingredient with a diluent and pouring the resulting mixture into soft or hard gelatin capsules.

A syrup or elixir preparation may contain the active ingredient together with a sweetener, an antiseptic, as well as a flavoring agent and a suitable colorant.

The water-dispersible powders or granules may contain the active ingredient by mixing with dispersing agents or wetting agents, or suspending agents, as well as with taste correctors or sweeteners. For intravenous administration, aqueous suspensions, isotonic saline solutions or sterile and injectable solutions which contain pharmacologically compatible dispersing agents and/or wetting agents are used.

The active ingredient may also be formulated as microcapsules, optionally with one or more additive carriers. Formulations suitable for the chosen form of administration are known to those skilled in the art and described, for example, in: Remington, The Science and Practice of Pharmacy, 22^{nd} Edition, 2013, The Pharmaceutical Press.

According to one embodiment, the present invention relates to the compound for its use according to the invention, said cancer being
head and neck cancer
lung cancer;
digestive cancer, such as rectal cancer, stomach cancer;
gynecological cancer such as uterine cancer, cervical cancer, vaginal cancer, ovarian cancer;
a urogenital cancer such as bladder cancer, prostate cancer, seminal vesicles, testes, germ cell tumors
ENT cancer such as cancer of the mouth, cheeks, palate, tongue, tonsils, pharynx, oropharynx and hypopharynx or cancer of the nasal cavity, sinuses, nasopharynx and larynx;
breast cancer
cancer of the small intestine;
colon cancer;
liver cancer;
cancer of the bile ducts;
gall bladder cancer;
pancreatic cancer;
kidney cancer;
cancers of the endocrine glands including thyroid cancer, pituitary gland, adrenal gland;
skin cancers including hemangiomas, melanomas, sarcomas, including Kaposi's sarcoma;
tumors of the brain, nerves, eyes, meninges, including astrocytomas, gliomas, glioblastomas, retinoblastomas, neuromas, neuroblastomas, schwannomas, meningiomas;
hematopoietic malignancies; leukemias, (Acute Lymphocytic Leukemia (ALL), Acute Myeloid Leukemia (AML), Chronic Myeloid Leukemia (CML), Chronic lymphocytic leukemia (CLL)) Chloromas, Plasmacytomas, T- or B-cell leukemias, Non-Hodgkin's or Hodgkin's lymphomas, Myeloma, various hematological malignancies;
any form of cancer at high risk metastatic.

According to one embodiment, the present invention relates to the compound of formula (I) for its use according to the invention, in combination with another anti-cancer agent, said other anti-cancer agent being a radiation, in particular a radiotherapy agent , said cancer being
head cancer
a cancer of the neck;
lung cancer;
a digestive cancer, such as a cancer of the rectum, a cancer of the stomach;
gynecological cancer such as uterine cancer, cervical cancer, vaginal cancer, ovarian cancer;
urogenital cancer such as bladder cancer, prostate cancer, seminal vesicles, testes, germ cell tumors;
ENT cancer such as cancer of the mouth, cheeks, palate, tongue, tonsils, pharynx, oropharynx and hypopharynx or cancer of the nasal cavity, sinuses, nasopharynx and larynx;
breast cancer
cancer of the small intestine;
colon cancer;
liver cancer;
bile duct cancer;
gall bladder cancer;
pancreatic cancer;
kidney cancer;
cancers of the endocrine glands including thyroid, pituitary, adrenal gland cancer;
skin cancers including hemangiomas, melanomas, sarcomas, including Kaposi's sarcoma;
tumors of the brain, nerves, eyes, meninges, including astrocytomas, gliomas, glioblastomas, retinoblastomas, neuromas, neuroblastomas, schwannomas, meningiomas; hematopoietic malignancies; leukemia, (Acute Lymphocytic Leukemia (ALL), Acute Myeloid Leukemia (AML), Chronic Myeloid Leukemia (CML), Chronic Lymphocytic Leukemia (CLL)) Chloromas, Plasmacytomas, T- or B-cell leukemias, Non-Hodgkin's or Hodgkin's lymphomas, Myeloma, various hematological malignancies;
any form of cancer at high risk metastatic

The present invention also relates to a process for separating the racemic form of the althiazide of formula (I), to obtain the enantiomers of formula (IA) and of formula (TB) in pure form, or with a chiral purity greater than 99%, by a supercritical fluid separation (SFC) technique, in particular at a temperature ranging from room temperature to 80° C. and in particular by SFC with an acid mobile phase or by SFC with a mobile phase comprising acetonitrile.

This separation mode makes it possible to obtain a high purity of the enantiomers (>99%), while limiting the enantiomer loss during the separation. For example, when separated with acetonitrile, the purities obtained are 100% for the first enantiomer (IA) and 99.4% for the second enantiomer (IB), with a mass recovery rate of the two enantiomers (IA) and (IB) 83.7%

The present invention also relates to a method of preserving enantiomers, to preserve their chiral purity, by storage at a maximum temperature of 5° C., and protected from light.

This mode of preservation makes it possible to preserve the chiral purity of the product, but does not make it possible to totally preserve the enantiomers of the althiazide from a degradation to another product than a form of althiazide.

The present invention also relates to a pharmaceutical composition containing a compound of formula (IA) or (IB) in combination with a pharmacologically acceptable carrier.

Glossary:
PSA (prostate gland specifies antigen): prostate specific antigen
AGM: aorta-gonad-mesonephros region
HSC: Hematopoietic stem cells CHT: Caudal hematopoietic tissue
GFP (Green Fluorescent Protein): Green Fluorescent Protein
NOEC: No Observed Effect Concentration: concentration at which the test compound is not effective
EC50 (Effective concentration 50%): concentration at which the test compound has an effectiveness of 50%
EHT: endothelial-to-hematopoietic transition
EMT (epithelial-mesenchymal transition): epithelial-mesenchymal transition
ACB: 4-amino-6-chloro-1,3-benzenedisulfonamide

### FIGURES

FIG. 1: Similarities of endothelial and epithelial cell migration processes at the EHT and the EMT. The main steps are conserved (loss of polarity and cell junctions, degradation and migration through the extracellular matrix, intravasation and extravasation into the bloodstream and colonization and proliferation in distant tissues).

FIG. 2: Observation of the exit and local migration of HSCs in the Aorta-Gonad-Mesonephros (AGM) region of the 48 hpf stage zebrafish embryo. After treatment with the thiazide at 10 μM, there is a reduction of the junction loss and the local migration of HSC.

FIG. 3: Percentage of cells accumulated in CHT after incubation of zebrafish embryos in the presence of molecules of the diuretic or antihypertensive therapeutic classes. Althiazide is the most effective chemical compound on inhibiting the migration of HSCs into distant tissues. Dasatinib is used as a reference molecule. The compounds are tested at the concentration of 10 μM.

FIG. 4: Validation of the effect of althiazide on the migration of HSCs in the zebrafish embryo in vivo. Decrease in the number of cells that migrated in CHT by 36.2% after treatment with althiazide by balancing at a concentration of 10 μM. The number of embryos is respectively n=64 and n=74. Error bar calculated with the standard error. Statistical test: student test. p-value: 2.9864E-7

FIG. 5: Comparison of the anti-micratory effects of dasatinib and althiazide on the zebrafish embryo in vivo. Both molecules are diluted at a concentration of 10 μM and tested by balancing on zebrafish embryos. The control solution contains an equivalent amount of DMSO (1%). In this test, the numbers of embryos are respectively n=80, n=79 and n=81. Error bar calculated with the standard error. Statistical test: student test. p-value: 5.6595E-6

FIG. 6: Curve of the dose effect of althiazide on the zebrafish embryo. Observation of the number of cells migrated in distant tissues (CHT) after treatment with balneation (percentage of effectiveness) with a solution containing althiazide at concentrations of 1 μM, 10 μM, 100 μM, 1000 μM. NOEC: No Observed Effect Concentration. EC50: Effective concentration 50%.

FIG. 7: Evaluation of the toxicity of althiazide and dasatinib on the zebrafish embryo by measuring the percentage of embryo survival as a function of the concentration of althiazide and dasatinib (A) or the duration of treatment (B). The molecules are diluted at concentrations of 1 μM, 10 μM, 100 μM and 1000 μM and incubated for 24, 48 and 72 hours. The control solution contains an equivalent amount of DMSO (1%). The number of embryos being respectively n=50, n=50 and n=50). Error bar calculated with the standard error. Statistical test: student test. p-value: 1.49852E-7.

FIG. 8: Migration and cytotoxicity test on 4T1 cells in culture in the presence of althiazide (A) or dasatinib (B) at concentrations of 10 nM, 100 nM, 1 μM, 10 μM and 100 μM. Error bar calculated with the standard error.

FIG. 9: Percentage of cells accumulated in CHT after incubation of zebrafish embryos in the presence of molecules of the class of thiazide compounds at different concentrations. Althiazide is the most effective chemical compound on inhibiting the migration of CD41 cells into distant tissues. The compounds are tested at a concentration of 10 μM or 25 μM.

FIG. 10: Rate of cells migrated during a migration test on MDA-MB-231 cells, as a function of the amount of althiazide added (10, 50 or 100 μM) compared to the control test.

FIG. 11: Impact of althiazide on the expression of transcription factors of genes involved in the epithelio-mesenchymal transition (EMT) under hypoxic conditions. The genes ZEB1, SNAIL, Vimentin and N-cad have a deleterious impact and must be inhibited. The E-cad gene has a beneficial effect and needs to be overexpressed. The white columns mention the control while the black columns are the expression results of these genes with an althiazide concentration of 100 μM.

FIG. 12: Cell viability and cytotoxicity test on MDA-MB-231 cells in the presence of doxorubicin alone (white columns) or of an althiazide and doxorubicin mixture (black columns), at different concentration (doxorubicin: 0.1, 0.5, 1, 2.5, 5, 10, 25, 50, 100 μM, althiazide: 100 μM).

FIG. 13: Cytotoxicity test on MDA-MB-231 cells in the presence of doxorubicin alone (Control) or of a mixture of doxorubicin supplemented with a compound of the invention at a concentration of 50 μM.

FIG. 14: Preparatory separation test between the two enantiomers of althiazide with 5 injections of 2 mL each. The line at the bottom of the peaks indicates the opening moments of the collection of each product. The first peak corresponds to the enantiomer AZ(A) and the second peak to the enantiomer AZ(B).

FIG. 15: Chromatogram of althiazide AZ-MI15 (racemic form). The first peak with a retention time of 10.15 minutes corresponds to the product AZ(A), and the second peak with a retention time of 13.4 minutes corresponds to the product AZ(B).

FIG. 16: Chromatogram of the sample EV-VZWOO 1-070-002. The first peak with a retention time of 10.2 minutes corresponds to the product AZ(A), and the second peak with a retention time of 13.5 minutes corresponds to the product AZ(B).

FIG. 17: Chromatogram sample EV-VZWOO 1-070-005 after 24 h storage under controlled temperature at 5° C. and protected from light. The first peak with a retention time of 10.09 minutes corresponds to the product AZ(A), and no peak near 13.5 minutes is visible.

FIG. 18: Chromatogram sample EV-VZWOO 1-070-006 after 24 h storage at room temperature without protection against light. The first peak with a retention time of 10.25 minutes corresponds to the product AZ(A), and the second peak with a retention time of 13.79 minutes corresponds to the product AZ(B).

### MATERIALS AND METHODS

The althiazide batch used is noted AZ-M015 and is in racemic form. The first enantiomer of formula (IA) and derived from AZ-M015 is noted AZ(A). The second enantiomer of formula (IB) and derived from AZ-M015 is noted AZ(B).

The analysis of the chiral purity of the products to be tested was carried out by a supercritical fluid separation (SFC) technique on a Berger prep chromatograph, marketed by Thar Instruments. The mobile phase of the separation consists of a mixture of methanol added. 0.01% acetic acid and CO₂ in a 20/80 ratio. The stationary phase chosen is a Chiralpak IC 59 m column, 250 mm by 20 mm, marketed by sigma aldrich. The injection rate is 50 ml per minute, at 40° C. under a pressure of 100 bar. The product to be analyzed is dissolved in methanol supplemented with 0.01% acetic acid, with a concentration of 1 mg/ml. 10 μl of this product is injected. The detector is a UV detector with a wavelength greater than 275 nm.

The analysis of the reverse phase purity of the products to be tested was carried out by an ultra-high pressure liquid chromatography (UHPLC) reverse phase chromatography technique on a Waters UPLC2 liquid chromatograph, marketed by Waters. The mobile phase of the separation varies during the method according to the protocol of Table 1.

TABLE 1
Composition of the mobile phase used for UHPLC analysis.
Between each point, the flow varies by gradient (TFA: Trifluoroacetate)
The column used is a Waters Acquity column UPLC HSC Cl 8
1.7 μm 2.1 * 100 mm, marketed by Water. The flow of mobile
phase is 0.6 ml/minute. The detector is a UV detector with
a wavelength of 210 nm. The product to be analyzed is dissolved
in acetonitrile, with a concentration of 1 mg/ml, and the
injected volume is 1 μl.

| | |
|---|---|
| Time | mobile phase (%) |

| (min) | water + 0.1% TFA | Acetonitrile |
|---|---|---|
| 0 | 95 | 5 |
| 4 | 5 | 95 |
| 5.5 | 5 | 95 |
| 5.51 | 95 | 5 |
| 7.3 | 95 | 5 |

The separation of the two enantiomers of the AZ-M015 AZ(A) and AZ(B) althiazide was carried out by a supercritical fluid separation (SFC) technique on a Berger prep chromatograph. The shift in the retention time of each enantiomer makes it possible to recover, at the separation outlet, first the compound AZ(A) and, secondly, the compound AZ(B). The recovery is indexed to the detector signal for opening and closing the recovery of each compound.

The mobile phase of the separation consists of a methanol mixture supplemented with 0.01% acetic acid and CO₂ in a 20/80 ratio or a mixture of acetonitrile and CO₂ in a 30/70 ratio. The stationary phase chosen is a Chiralpak IC 5 μm column, 250 mm by 20 mm. The injection rate is 50 ml per minute, at 40° C. under a pressure of 100 bar. The product to be separated is dissolved in a methanol mixture supplemented with 0.01% acetic acid or in acetonitrile. A series of injections is performed. The detector is a UV detector with a wavelength greater than 275 nm. The collection of the respective products is started when the signal exceeds a high threshold detection value and is stopped when the signal falls below a low threshold detection value. The high and low threshold values are chosen during a preparatory test according to a compromise between the quantity of enantiomers recovered, the desired purity and the analysis conditions. The collected products are evaporated before being analyzed for their respective purity.

Solubilization of althiazide and enantiomers was achieved by solubilization in a solvent of 95% 1M Tris pH 10.8 and supplemented with 5% pure ethanol.

Althiazide and enantiomers are resuspended at a concentration of 2 mg/ml. Each of the solutions is vortexed for 30 seconds and then placed in an ultrasound bath for a sonication of 5min; this operation is repeated once. The dissolution is complete despite the absence of DMSO.

The solubilization of althiazide and enantiomers was also obtained by solubilization in a solution consisting of a mixture of 3.7% of NaHCO₃, 0.2M and 21% of Na₂CO₃ 0.2M at pH 10.6 and supplemented with pure water. Althiazide and enantiomers are resuspended at a concentration of 2.5 mg/ml. Each of the solutions is vortexed for 30 seconds and then placed in an ultrasound bath for a sonication of 1 min. The dissolution is complete despite the absence of DMSO.

The stability tests of the enantiomers were performed by comparing the racemization and the degradation of the stored samples either at room temperature without protection against light, or under controlled temperature at 5° C. and protected from light.

The stored products are analyzed regularly (T=0, 1 h, 2 h, 3 h, 6 h, 24 h) to follow the evolution of the racemization of enantiomers.

The Tg transgenic zebrafish line (CD41:GFP) is maintained in accordance with the protocols described by the Ethical Committee for Animal Experimentation. The fish are kept at a temperature of 28° C. and their development stage is determined as before. The molecules are tested on zebrafish embryos at the 25 hpf stage. The embryos are decorticated and transferred to a 96-well plate (1 embryo/well) containing the test compounds in a final volume of 100 μl of the embryonic growth medium (60 μg salt for 1 ml H2O). For chemical compounds diluted in DMSO, the final concentration of DMSO does not exceed 1%. The treated embryos are incubated for 24 hours in an incubator at 28° C.

The embryos are imaged with a plate reader after being anesthetized with 0.16% tricaine (ethyl-3-aminobenzoate). A quantification of the number of fluorescent cells (CD41:GFP) accumulated in the CHT is carried out.

The toxicity study is carried out over 72 hours. The embryos are treated at 25 hpf with the doses used for the test of chemical compounds of each of the compounds, in a 96-well plate, in a final volume of 100 μl of the embryonic growth medium. The embryos are incubated 72 h at 28° C. The medium is changed every 24 hours. The survival rate of embryos and the appearance of developmental defects are analyzed daily.

The cell migration assays on the 4T1 cell line are carried out in vitro in 12-well plates on the 4T1 cell line. The cells are maintained in DMEM culture medium supplemented with 10% calf serum (FCS) and incubated at 37° C. and 5% CO₂. The cell monolayer is injured with a 10 μ pipette tip. The medium is aspirated and replaced by a medium containing the compounds to be tested at the concentrations described. The wells are imaged using a microscope and the injured surface is repaired at the indicated times to highlight the cell anti-migratory power of the tested chemical compounds. The average of the measurements obtained is compared with the control wells: (average value of the compounds treated/average value of the controls)*100=% of the average value.

Cell migration assays on the MDA-MB-231 cell line are performed in vitro in 96-well plates. The MDA-MB-231 cells were seeded at the density of 50000 cells per well, to form a monolayer. The cells are maintained in a DMEM culture medium supplemented with 0.2% fetal calf serum and incubated at 37° C., 5% CO₂ and 1% O₂ for 24 hours. The cell monolayer is injured with a pipette tip. The cells are then rinsed with PB S to remove the cells in suspension and the different treatments are added, as well as DMEM supplemented with 10% FCS. The cells are incubated for 18 hours at 37° C., 5% CO₂ and 20% O₂.

A first series of images of each well (center of the well) was carried out at the beginning of the experiment (t=0) and after 18 hours of migration (t=18) using a 4× objective Nikon microscope. The images were analyzed using ImageJ software (National Institutes of Health, USA).

The treatments were carried out in triplicate and the manipulation was carried out 3 times.

The cytotoxicity tests are carried out in cell culture in 96-well plates, on the 4T1 cell line. The cells are maintained in a DMEM culture medium supplemented with 10% fetal calf serum (control condition) or placed in the presence of an MTT solution (3-(4,5-dimethylthiazol-2-yl)-2,5-diphenyl tetrazolium bromide) at 1 mg/ml in DMEM medium supplemented with 10% fetal calf serum (100 μl per well). The cells are incubated at 37° C. and 5% CO₂ for 4 hours. The reaction is stopped by the addition of 100 μl of a solution containing 10% of SDS and 0.01 M of HCl. The cells are placed in an incubator at 37° C. and 5% CO₂ for 2 hours. The absorbance is measured between 570 and 590 nm. The negative control is performed on a well without cells, containing 200 μl of a 1 mg/ml solution of MTT diluted in DMEM culture medium supplemented with 10% fetal calf serum. The average of the measurements obtained is compared with the control wells: (average value of the compounds treated/average value of the controls)*100=% of the average value.

The cell cultures are carried out on human breast cancer epithelial cell lines MDA-MB-231 (HTB-26™), resulting from metastases present in a pleural effusion of a patient with a mammary adenocarcinoma. This line is used as a triple negative cancer cell model (no expression of ER-type nuclear estrogen receptors, nuclear progesterone receptors or overexpression of the oncogene encoding HER2 protein (Human Epidermal Growth factor Receptor-2).

The cells were cultured in DMEM medium (Eurobio, Fance) supplemented with 10% fetal calf serum (FCS) (Eurobio, Fance) at 37° C., 5% CO₂. The medium was changed every 3 days.

The cells were placed in a controlled atmosphere at 1% oxygen 24 hours before the application of the different treatments.

Proliferation is measured by crystal violet staining. Cells were seeded in 96-well plates at a density of 10,000 cells per well. After 24 h of culture at 37° C., 5% CO₂ and 1% O₂, the medium was renewed and added with althiazide (at the concentrations described). Cells were incubated for an additional 24, 48 or 72 hours at 37° C., 5% CO₂ and 1% O₂. After rinsing the cells with PBS, 50 μl of a 0.5% crystal violet solution in 20% ethanol was added to each well. The plate was incubated at room temperature with stirring for 15 minutes. The cells were washed and then lysed with a 1% SDS solution in order to solubilize the crystal violet. Absorbance was measured at 570 nm using the Tecan Sunrise spectrophotometer.

The results are expressed as a percentage of the control.

The cell viability is determined via the IC50 index, measured by the MTT technique (2-(4,5-dimethyl-2-thiazolyl)-3,5-diphenyl-2H-tetrazolium bromide) (Mosmann, 1983). This colorimetric test is based on the reduction of yellow tetrazolium salts into insoluble purple formazan crystals in aqueous solution. The spectrophotometric measurement of the absorbance at 570 nm is directly proportional to the number of viable cells

The cells were seeded in 96-well plates at a density of 25,000 cells per well. After 24 h of culture at 37° C., 5% CO₂ and 1% O₂, the medium was renewed and supplemented with increasing doses of doxorubicin+/−althiazide. Cells were incubated for 24 h at 37° C., 5% CO₂ and 1% O₂. The MDA-MB-231 cells were washed and then incubated for 1 hour at 37° C. with 100 μl of a 0.5 mg/ml MTT solution prepared in culture medium. The formazan crystals were dissolved in 100 of DMSO and the absorbance measured at 570 nm using the Tecan Sunrise spectrophotometer. The IC50 was determined using the Graphpad Prism 5.0 software. The treatments were carried out in triplicate and the manipulation was carried out 5 times.

The expression of the mRNAs is measured by the q-RT-PCR method. The cells were seeded at the density of 500,000 cells/well in 6-well plates. After 24 hours at 37° C., 5% CO₂ and 1% O₂, the medium was renewed and added the various treatments. RNA was isolated using TRI Reagent® (sigma), assayed and retro-transcribed into complementary DNA using the PrimeScript RT Reagent Kit (Takara). The expression levels of the mRNA of the genes of interest were determined by quantitative PCR (LightCycler® 480 Instrument II) with the primers listed in Table 2:

TABLE 2
List of primers used by qPCR

| | | |
|---|---|---|
| hactin_for | CCAACCGCGAGAAGATGACC | SEQ ID NO: 1 |
| hactin_rev | GATCTTCATGAGGTAGTCAGT | SEQ ID NO: 2 |
| hZEB1_for | GATGATGAATGCGAGTCAGATGC | SEQ ID NO: 3 |
| hZEB1_rev | CTGGTCCTCTTCAGGTGCC | SEQ ID NO: 4 |
| hSLUG_for | GCT CCT TCG TCC TTC TCC TC | SEQ ID NO: 5 |
| hSLUG_rev | TGA CAT CTG AGT GGG TCT GG | SEQ ID NO: 6 |
| hSNAIL_for | GGCCTTCAACTGCAAATACT | SEQ ID NO: 7 |
| hSNAIL_rev | ACATCTGAGTGGGTCTGGAG | SEQ ID NO: 8 |
| hVimentin_for | GACAATGCGTCTCTGGCACGTCTT | SEQ ID NO: 9 |
| hVimentin_rev | TCCTCCGCCTCCTGCAGGTTCTT | SEQ ID NO: 10 |
| hE-cadh_for | CCCACCACGTACAAGGGTC | SEQ ID NO: 11 |
| hE-cadh_rev | CTGGGGTATTGGGGGCATC | SEQ ID NO: 12 |
| hN-cadh_for | GCCACCTACAAAGGCAGAA | SEQ ID NO: 13 |
| hN-cadh_rev | ATGTGCCCTCAAATGAAACC | SEQ ID NO: 14 |

The results are calculated using the Delta Delta Ct (Δ ΔCt) method.

The anti-metastatic effects of althiazide in mice are evaluated in immunocompromised mice injected with cells from a tumor line in the mammary gland.

The mice are treated for 4 weeks with an althiazide injection each day. The mammary tumors of the mice are measured 3 times a week for 4 weeks.

Mice for which the mammary tumor has reached 2 g are sacrificed and the number of cancerous metastases invading the lung is counted.

This number is compared to the batch of control mice injected with the same tumor cell line and treated with PBS. A decrease in the number of metastases in the lungs of mice treated with althiazide compared to the control lot in which the mice are treated with PBS makes it possible to validate the anti-metastatic effect of althiazide in vivo in mice.

The potentiating effects of althiazide on an anti-cancer agent in mice are evaluated in immunocompromised mice injected with cells from a tumor line in the mammary gland.

The maximum dose of althiazide is given alone or in combination with the anti-cancer agent. Several doses of anti-cancer agent are tested.

The mice undergo a treatment for 4 weeks with an injection of anticancer agent per week over 4 weeks and an injection of althiazide every day for 4 weeks. The first dose of althiazide is given on the first day after injection of cells from a tumor line into the mammary gland.

The mammary tumors of the mice are measured 3 times a week and compared to the batches of mice treated with PBS alone or with althiazide alone.

A reduction in the size of cancerous tumors in the lot of mice treated with althiazide and doxorubicin compared to the control lot or the mice are treated with doxorubicin alone, to validate the potentiating effect of althiazide on the activity of doxorubicin in vivo in mice.

### EXAMPLES

### Example 1: Anti-Metastatic Action of Althiazide: Test of Initiation and Progression of the Metastatic Process on the Zebrafish Embryo

Althiazide was tested on the zebrafish embryo according to the method previously described in the international application WO 2011/007259. This method consists in isolating molecules capable of inhibiting the emergence of hematopoietic stem cells from the floor of the aorta without cell division, but by endothelial-to-hematopoietic transition. This emergence involves the loss of cell polarity of emerging cells, the loss of cellular junctions with these neighboring cells and the acquisition of migratory properties. This model is used, in the context of the invention, because it mimics each of the stages of the epithelial-to-mesenchymal transition, the first stage of the cancerous metastasis. The endothelial-to-hematopoietic transition takes place in a region of the embryo called Aorto-Gonado-Mesonephros (AGM).

The colonization of hematopoietic tissues by hematopoietic stem cells mimics the appearance and/or progression of a metastatic tumor since it involves intravasation, extravasation, or the migration of HSCs following a gradient of SDF1. The first colonized tissue is caudal hematopoietic tissue (CHT). This physiological process is considered, in the context of the invention, as representative of the appearance and/or progression of a metastatic tumor.

In order to evaluate the inhibition of endothelial-to-hematopoietic transition, CD41: green fluorescent protein (GFP) transgenic embryos, in which hematopoietic stem cells (HSCs) express GFP, were used, allowing them to be monitored in vivo under a fluorescence microscope.

Each CD41:GFP embryo at the 25-hour post fertilization (hpf) stage was added to a well of a 96-well plate containing one of the compounds to be tested. The treated embryos were incubated 24 hours at 28° C.

Each well containing the 50 hpf embryos was then imaged using a fluorescence microscope to analyze the number of cells dispersed in the embryo and to quantify their distribution in the different regions of the embryo.

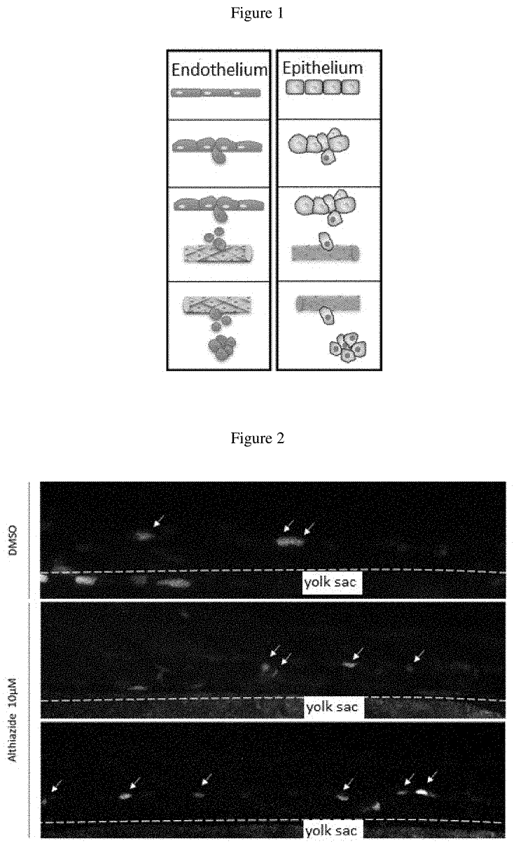

The read-out is the number of hematopoietic stem cells CD41:GFP accumulated in the AGM (mimicking the initiation of the metastatic process) and/or in the CHT of the zebrafish embryo (mimicking the progression of the metastatic process). The accumulation of CD41:GFP cells in CHT implies that each cell has made an endothelial-to-hematopoietic transition, an intravasation, a CHT extravasation, has settled in a stromal niche and has proliferated (FIG. 1). In parallel, the phenotypic characterization of embryos has made it possible to isolate compounds capable of preventing the endothelial-to-hematopoietic transition and their migration towards the CHT without causing a toxic effect on the developing embryo.

According to the methodology described above, althiazide has proved effective against the migration of HSC locally and to distant organs (CHT).

After treatment with the concentration of 10 μl of althiazide (concentration in the embryonic growth medium: 60 g of salt in 1 ml of distilled H2O), the number of HSC accumulated in the AGM is greater than the number of HSC accumulated in reference embryos treated with the althiazide dissolution solution, DMSO (FIG. 2). Althiazide reduces the emergence of HSCs and their local migration.

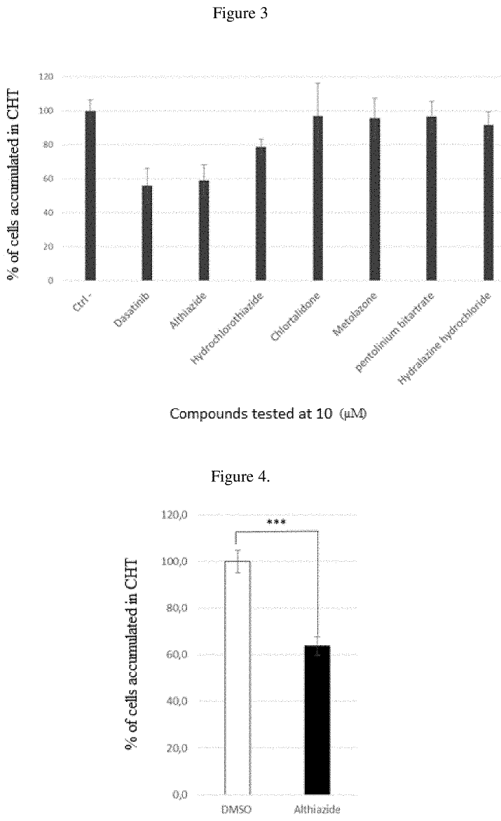

After treatment with a concentration of 10 μl of althiazide (concentration in the embryonic growth medium: 60 μg of salt in 1 ml of distilled H₂O), the number of HSCs accumulated in CHT is reduced by 41% compared with number of HSC accumulated in reference embryos treated with the solution of dissolution of althiazide, DMSO (FIG. 3), and this without causing toxic effects. This effect is identical to that observed with dasatinib, a reference molecule in this test, which has been used in various clinical trials for its potential anti-metastatic effects. Although the anti-metastatic effects of dasatinib have been observed in various experimental models, it has not been used as an anti-metastatic treatment in humans, mainly because of its low tolerability (fatigue, hyponatremia, diarrhea, gastrointestinal bleeding, pleural and pericardial effusions and anemia) by the patients. Dasatinib is a potent inhibitor of many kinase enzymes such as BCR-ABL, KIT and PDGFRα/β, and is therefore primarily used for the treatment of chronic myeloid leukemia resistant to imatinib, but also a potent inhibitor of c-SRC, LYN, FY and EPHA2, which may explain its low tolerability.

### Example 2 Absence of Anti-Metastatic Effect of Other Diuretics or Antihypertensives: Evaluation of the Progression of the Metastatic Process on the Zebrafish Embryo

In order to verify the specificity of the action of althiazide, other chemical molecules of the therapeutic classes of diuretic or anti-hypertensive (Chlortalidone, Metolazone, Pentolinium-bitartrate and Hydralazine-hydrochloride) were tested at the same concentration than althiazide (10 μM). These chemical molecules do not cause a decrease in the number of HSCs accumulated in CHT. A decrease of 20% is however observed after treatment with the concentration of 10 μM of Hydrochlorothiazide which has a structure close to althiazide.

A second test, on a larger number of embryos, confirmed the efficacy of althiazide as an anti-metastatic with a decrease in the number of cells colonized CHT by 36.2% (FIG. 4).

### Example 3: In Vivo Anti-Migration Activity: Test of the Progression of the in Vivo Metastatic Process on the Zebrafish Embryo

Considering that an anti-migratory effect is a good indicator of antimetastatic activity, we assessed the anti-migratory effect of althiazide in vivo on the zebrafish embryo.

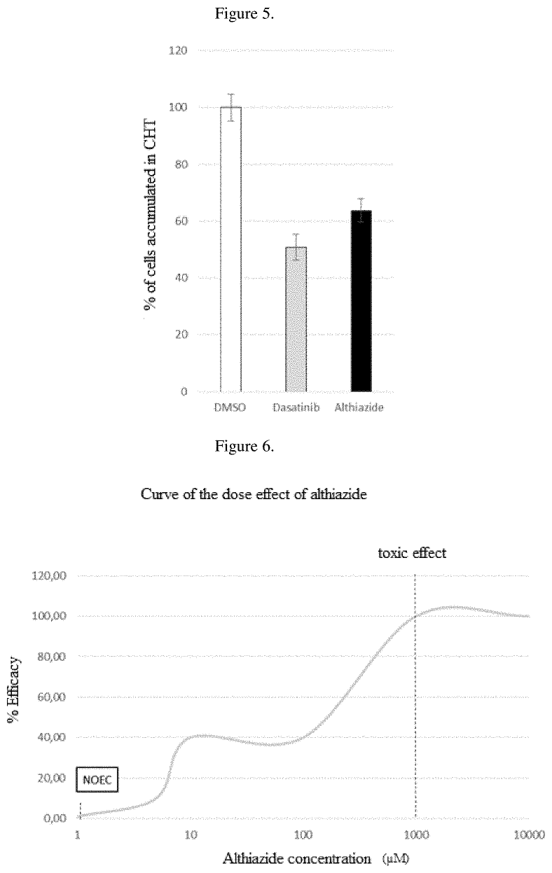

The number of HSCs accumulated in the distant tissues (CHT) of the zebrafish embryo after treatment with the 10 μM concentration of althiazide and dasatinib, the reference molecule in this test, was compared. We concluded that althiazide has an anti-migratory effect as effective as dasatinib, used at the same concentration (FIG. 5).

### Example 4: Measurement of the Effectiveness Threshold of the Althiazide: Effectiveness/in Vivo Toxicity Dose Test on the Zebrafish Embryo

In order to determine the concentration at which the althiazide is not effective (NOEC), the concentration at which the althiazide is 50% effective (EC50) and the concentration at which the althiazide is 100% effective, the number of cells migrated to distant tissues (CHT) after balancing with a solution containing althiazide at concentrations of 1 μM, 10 μM, 100 μM, 1000 μM for 24 hours was quantified.

At 1 μM, no effect is observed, at 10 μM, an efficiency of 50% is observed, at 100 μM, an efficiency of 100% is observed but also the appearance of toxic effects (FIG. 6).

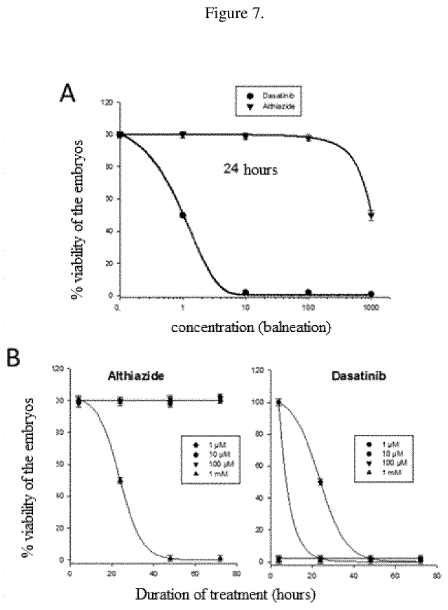

At the same time, we analyzed the toxicity of althiazide after balancing with a solution containing althiazide at concentrations of 1 μM, 10 μM, 100 μM, 1000 μM for 24, 48 and 72 hours. The toxicity study was based on analysis of the percentage viability of the embryos after treatment. At concentrations of 1 μM, 10 μM and 100 μM, althiazide causes no toxicity even after 72 hours of treatment. A 50% toxicity is observed after treatment for 24 hours, the althiazide at the concentration of 1 mM. In comparison with the doses evaluated with althiazide, dasatinib has a very high toxicity (FIG. 7).

### Example 5: Cytotoxicity: Cytotoxicity Test on 4T1 Cells

The 4T1 cells are derived from tumors of the mammary gland in mice.

It is a particularly suitable model and widely used for studies on cancerous tumors and more particularly on metastasis. Indeed, mammalian in vivo efficacy studies are performed on the mouse and the 4T1 model is an orthotopic syngeneic model of murine cells injected into BalbC mice, which promotes dialogue between the tumor and its environment; essential condition for cell migration. Moreover, this model is known to develop a large number of metastases in a relatively short period of time. Metastases appear in the lungs, which facilitates their observation in the visible.

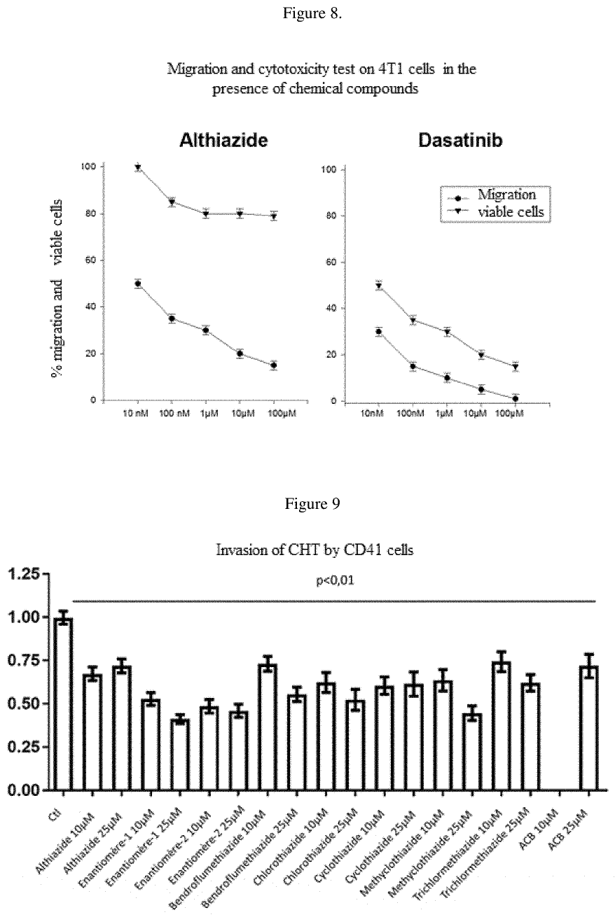

In order to study the cytotoxic effects of althiazide in vitro, we analyze the cell death induced by treatment with althiazide by tetrazolium salt labeling MTT (3-(4,5-dimethylthiazol-2-yl)-2,5-diphenyltetrazolium bromide) on 4T1 cells. The amount of living cells in the well is assayed by spectrometry. The results are compared with those obtained after treatment with the control solution (FIG. 8). In this test, we observe a very low mortality of cells after treatment with althiazide (80% of living cells at the highest dose 100 μM). Cells treated with dasatinib have a much higher mortality rate, at much lower doses (80% live cells at the lowest 10 nM dose and 20% live cells at 100 μM).

### Example 6: Anti-Metastatic Activity of Althiazide in Vitro: 4T1 Cell Migration Test

In order to validate the anti-migratory potential of althiazide, we carry out a “scratch test” on 4T1 cells in culture according to the cell migration test protocol on the 4T1 cell line described in the Materials and Methods section. This test is used to evaluate the migratory power of a chemical compound on cells in culture. The experiment consists in studying the capacity of cells to reconstitute a cellular mat (‘cicatrisation’), which implies the migration property of the cells composing the cell monolayer on the injured area. The test is carried out in a 12-well microtiter plate. An injury is performed using a pipette tip of 10 μl, then the cells are treated with dasatinib or althiazide at concentrations of 10 nM, 100 nM, 1 μM, 10 μM and 100 μM. The cells are then placed under normoxic or hypoxic conditions. A photo of the surface of the well is made at t=0 (after the injury) then at t=24 h.

The area of the injured area is measured for each well and the percentage of migration is calculated under each condition.

We observe a decrease in cell migration of 40% after treatment with althiazide at the lowest concentration 10 nM (FIG. 8). At the same doses, dasatinib is more effective but causes much more toxicity.

### Example 7: Anti-Migratory Action of Other Thiazide Compounds: Test for Initiation and Progression of the Migratory Process on the Zebrafish Embryo

To determine whether the anti-metastatic action of althiazide is also valid for the entire thiazide family, other thiazide compounds were tested.

The anti-metastatic efficacy of the thiazide compounds was determined as for the measurement of the anti-metastatic efficacy of athiazide, quantified by the cell migration assay on the 4T1 cell line described in the Materials and Methods section. These compounds were tested at 10 and 25 μM. Each of these compounds makes it possible to reduce the number of cells having colonized CHT (caudal haematopoietic tissue) and thus confirms the anti-metastatic activity of the thiazide compounds. AZ-M015 reduces migration by 30%. However, the most effective compound is MCZ since it reduces migration by 37% when used at 10 μM and 55% when used at 25 μM. In addition, the enantiomers of PAZ-M015, AZ(A) and AZ(B) further reduce the invasion of CHT by CD41:GFP cells. These compounds, by decreasing the accumulation of CD41:GFP cells, altered one or more of the following processes: endothelial-to-hematopoietic transition, intravasation and/or extravasation at the CHT level.

### Example 8: Migration Test MDA-MB-231 Cells

In order to validate the anti-migratory potential of althiazide, we carry out a “scratch test” is carried out on MDA-MB-231 cells in culture according to the protocol of the cell migration test on the MDA-MB-231 cell line described in the Materials and Methods section. This test is used to evaluate the migratory power of a chemical compound on cells in culture. The experiment consists in studying the capacity of cells to reconstitute a cellular mat (‘cicatrisation’), which implies the migration property of the cells composing the cell monolayer on the injured area. The test is carried out in a 96-well microtiter plate. Injury is performed using a pipette tip, and then the cells are treated with althiazide.

A first series of images of each well (center of the well) is performed at the beginning of the experiment (t=0) and after 18 h of migration (t=18) using a Nikon microscope −4× lens. The images were analyzed using Image J software (National Institute of Health, USA)

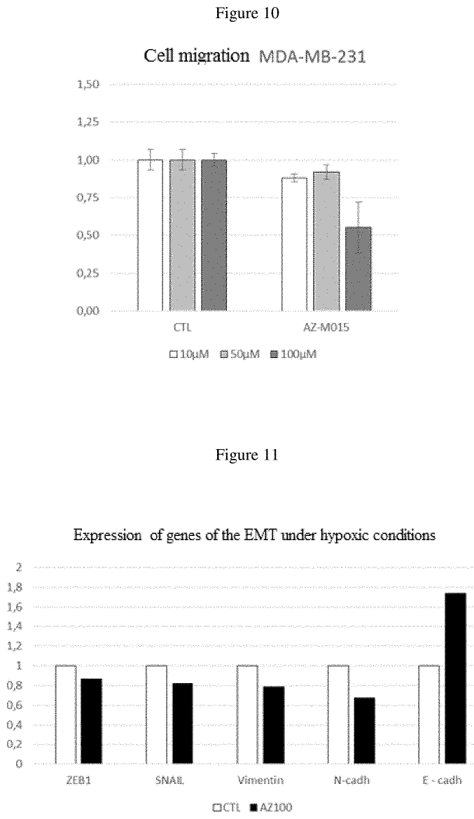

An anti-migratory activity of AZ-M015 is observed at concentrations of 10, 50 and 100 μM. A decrease in the order of 50% of the migration is observed (FIG. 10).

### Example 9: Expression of Markers of the Epithelio-Mesenchymal Transition

The epithelial-mesenchymal transition (EMT) is the first step in the process of formation of cancer metastases. Indeed, epithelial cells begin by performing a EMT (loss of cellular adhesion, loss of cellular polarity and acquisition of migratory properties) and then become invasive (Thiery & Sleeman, 2006).

TEM is characterized by overexpression of transcription factors

SNAIL/SLUG/TWIST/ZEB1 that induce gene reprogramming leading to the acquisition of migratory properties, in particular the increase in the expression of VIMENTINE and N-CADHERINE and the decrease in the expression of E-CADHERIN.

Here, AZ-M015 induces a decrease in the expression of transcription factors ZEB1 (−14%) and SNAIL (−19%), VIMENTIN (−22%) and N-CADHERIN (−33%) as well as an increase in FE-CADHERIN expression of 73% (FIG. 11).

These modifications are opposite to those observed during the TEM. AZ-M015 is therefore an inhibitor of the TEM. By this inhibition, AZ-M015 acts directly on the metastatic spread by blocking the first stage of this cancerous process.

### Example 10: Cytotoxicity Potentiation Tests of Althiazide

Doxorubicin is a substance widely used in the treatment of many cancers (bronchopulmonary cancers, stomach cancers, ovarian cancers, bladder cancers, breast cancers, leukemias, multiple myeloma, non-Hodgkin's malignant lymphoma, Hodgkin's disease, AIDS-associated Kaposi's sarcomas, bone sarcomas, soft tissue sarcomas and solid tumors in children). The use of anthracyclines and in particular doxorubicin is associated with a risk of cardiotoxicity in a dose-dependent manner that can progress to severe and irreversible heart failure. Thus it is on the one hand, important to improve the effectiveness of this treatment and on the other hand to reduce toxicity. In this test, althiazide was added to doxorubicin and cell viability was then determined.

The tests are carried out in cell culture in 96-well plates, on the cell line MDA-MB-231. The MDA-MB-231 cells are seeded in 96-well plates at a density of 10,000 cells and incubated 24 h at 37° C., 5% CO₂ and 1% O₂. The medium is then renewed and added with increasing concentrations of doxorubicin+/−100 μM althiazide. The cells are incubated for an additional 24 hours at 37° C., 5% CO₂ and 1% O₂. Treatments were made in triplicas and the experiment was repeated 5 times.

A cell viability test is then performed according to the method described in Materials and methods.

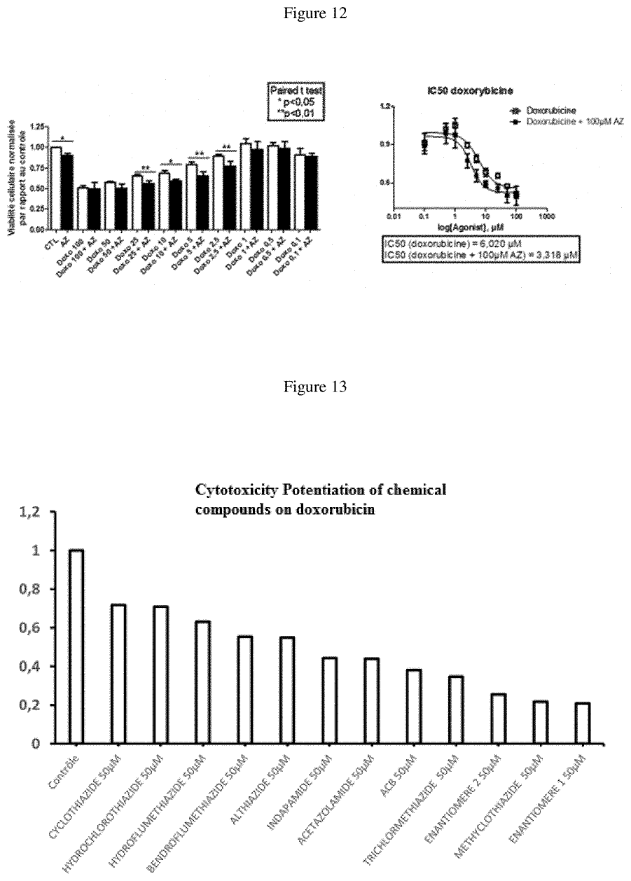

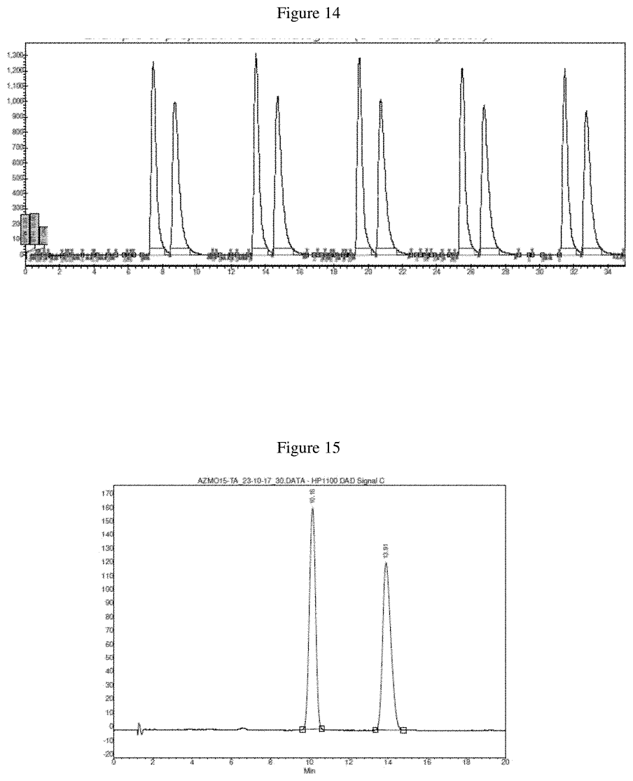

Results are normalized to the control condition (FIG. 12)

Althiazide increases the effects of doxorubicin when it is used between 25 and 2.5 μM of about 15%. In addition, treatment with athiazide reduces the IC50 of doxorubicin by 45% from 6.02 μM to 3.318 μM. Althiazide is therefore a potentiator of the anticancer agent doxorubicin. Thus, AZ-M015 makes it possible to increase the effectiveness of anti-cancer treatments and/or to reduce the dose used and thus reduce the undesirable toxic effects of this anti-cancer drug.

### Example 11: Cytotoxicity Potentiation Tests for Althiazide

The tests are carried out in cell culture in 96-well plates, on the cell line MDA-MB-231. The MDA-MB-231 cells are seeded in 96-well plates at a density of 10,000 cells and incubated 24 h at 37° C., 5% CO₂ and 1% O₂. The medium is then renewed and added with doxorubicin and another compound of the invention (Formula (IA, IB, IVa, Va, Vb, Vc, Vd, Ve, Vf, VIa, VIIa and VIIb) at a concentration of 50 μl. The cells are incubated for an additional 24 hours at 37° C., 5% CO₂ and 1% O₂. Treatments were made in triplicas and the experiment was repeated 5 times.

### Example 12: Evaluation of the Anti-Metastatic Effects of Althiazide on the Mouse

The anti-metastatic effects of althiazide on the mouse are evaluated in immunocompromised mice injected with MDA-MB-231 cells into the mammary gland.

The mice are treated for 4 weeks with an althiazide injection each day.

The mammary tumors of the mice are measured 3 times a week for 4 weeks.

Mice for which the mammary tumor has reached 2 g are sacrificed and the number of cancerous metastases invading the lung is counted. This number is compared to the lot of control mice injected with the MDA-MB-231 cancer cells and treated with PBS.

### Example 13: Evaluation of the Potentiating Effects of Althiazide on Doxorubicin in Mice

The maximum dose of althiazide is given alone or in combination with doxorubicin. 4 doses of doxorubicin are tested.

MDA-MB-231 cells are injected into the mammary gland of immuno-compromised mice.

The mice are treated for 4 weeks at a dose of doxorubicin per week over 4 weeks and an injection of althiazide every day for 4 weeks. The first dose of althiazide is given on the first day after injection of MDA-MB-231 cells into the mammary gland.

The mammary tumors of the mice are measured 3 times a week and compared to the batches of mice treated with PBS alone or with althiazide alone.

### Example 14: Separation of Enantiomers from Althiazide AZ-M015

a. Separation by SFC Acid

The separation of the two enantiomers was carried out according to the protocol described in the Materials and Methods section, with the following parameters:
Mobile phase: methanol supplemented with 0.01% acetic acid/CO₂ in a ratio 20/80
Product to be separated: 49.2 mg of AZ-M015 dissolved in 2 ml of methanol supplemented with 0.01% of acetic acid.
Injection: 10 injections of 5 mg each.
Collection: 50 mUA opening threshold, 30 mUA closing threshold.

The two compounds obtained following this separation were called EV-VZW001-070-001 (19.8 mg) and EV-VZW001-070-002 (20.1 mg).

b. Separation by SFC Acetonitrile Version 1

The separation of the two enantiomers was carried out according to the protocol described in the Materials and Methods section, with the following parameters:
Mobile phase: acetonitrile/CO₂ in a ratio 30/70
Product to be separated: 50.9 mg of AZ-M015 dissolved in 2 ml of acetonitrile.
Injection: 13 injections of 4 mg each.
Collection: Opening threshold 60 mUA, closing threshold 50 mUA.

The two compounds obtained following this separation were called EV-VZW001-070-003 (21.7 mg) and EV-VZW001-070-004 (22.1 mg). FIG. 14 shows the preparative tests for adjusting the opening and closing of the collection of each enantiomer. This collection mode necessarily leads to a lower purity of the second enantiomer.

c. Separation by SFC Acetonitrile Version 2

The separation of the two enantiomers was carried out according to the protocol described in the Materials and Methods section, with the following parameters:
Mobile phase: acetonitrile/CO₂ in a ratio 30/70
Product to be separated: 74.9 mg of AZ-M015 dissolved in 4 ml of acetonitrile.
Injection: 20 injections of 3.8 mg each.
Collection: 50 mUA opening threshold, 40 mUA closing threshold.

The two compounds obtained following this separation were called EV-VZW001-070-005 (33.6 mg) and EV-VZW001-070-006 (29.1 mg).

### Example 15: Analysis of the Separation

a. Analysis of the starting product.

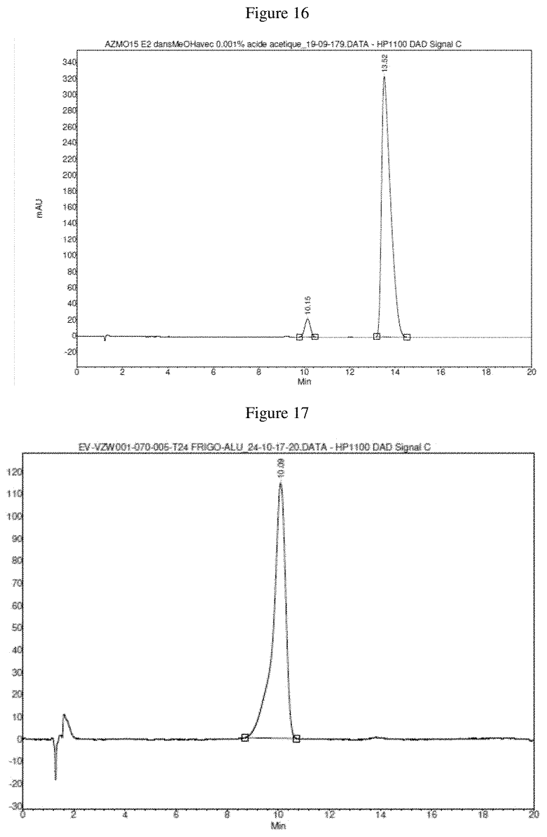

The starting compound, AZ-M015 was analyzed to identify the distribution of the two enantiomers by the chiral purity analysis protocol described in the Materials and Methods section. FIG. 15 shows the chromatograph that was obtained with a 49.8% distribution of AZ enantiomer (A) and 50.2% AZ enantiomer (B). b. Analysis of other compounds EV-VZW001-070-X.

Table 3 shows the results obtained for the compounds from the three separations according to the chiral purity analysis and reverse phase purity analysis protocols both described in the Materials and Methods section.

TABLE 3
Result of analysis of the compounds resulting from the separations.

| | UHPLC | SFC | |
|---|---|---|---|
| Composé | RP (%) | AZ(A) (%) | AZ(B) (%) |
| AZ-MO15 | 100 | 49.8 | 50.2 |
| EV-VZWOO1-070-001 | 95.6 | 95.5 | 4.5 |
| EV-VZWOO1-070-002 | 95.8 | 4.2 | 95.8 |
| EV-VZWOO1-070-003 | 99.5 | 99.7 | 0.3 |
| EV-VZWOO1-070-004 | 99.4 | 0.9 | 99.1 |
| EV-VZWOO1-070-005 | 99.8 | 100 | 0 |
| EV-VZWOO1-070-006 | 100 | 0.6 | 99.4 |

FIG. 16 shows the chromatogram obtained for the analysis of the sample EV-VZWOO 1 -070-002. Example 16: Stability test of enantiomers

After separation by SFC acetonitrile version 2, 1.5 mg of EV-VZWOO 1-070-005 and 2.4 mg of EV-VZWOO 1-070-006 are removed for stability testing.

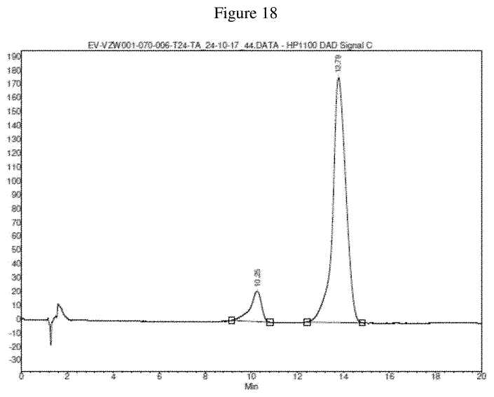

Sample EV-VZWOO 1-070-005 is stored at a temperature of 5° C. under light protection conditions. Sample EV-VZWOO 1-070-006 is stored at room temperature on the bench without protection against light. FIG. 17 shows the chromatogram obtained during the analysis of sample EV-VZWOO 1-070-005 after 24 h storage.

FIG. 18 shows the chromatogram obtained during the analysis of sample EV-VZWOO 1-070-006 after 24 hours of storage. Table 4 shows the evolution over time of the quantities of enantiomer present in the samples.

TABLE 4
Temporal stability of enantiomers stored or not in optimal conditions

| | SFC | | | | UHPLC | |
|---|---|---|---|---|---|---|
| | EV-VZWOO1- | | EV-VZWOO1- | | Pureté en phase inverse | |
| Temps | 070-005 | | 070-006 | | EV-VZWOO1- | EV-VZWOO1- |
| (en h) | AZ(A) (%) | AZ(B) (%) | AZ(A) (%) | AZ(B) (%) | 070-005 | 070-006 |
| 0 | 100 | 0 | 0.6 | 99.4 | 99.9 | 100 |
| 1 | 100 | 0 | 1.2 | 98.8 | / | / |
| 2 | 100 | 0 | 1.2 | 98.8 | / | / |
| 3 | 100 | 0 | 1.7 | 98.3 | / | / |
| 6 | 100 | 0 | 3 | 97 | / | / |
| 24 | 100 | 0 | 9.7 | 90.3 | 94.9 | 90.5 |

1.-21. (canceled)

22. Method of treatment of cancer comprising administering to a subject a compound of formula (II) 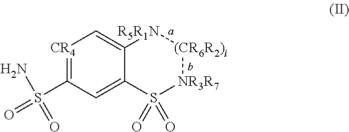
in which:
a can represent:
nothing,
a single bond,
a double bond;
b represents:
nothing if a represents nothing,
a single bond if a is either a single bond or a double bond;
the value of i being:
zero if a represents nothing,
equal to 1 if a is a single or a double bond;
if a represents nothing, then b represents nothing and i is 0:
R₁ and R₅ can represent independently of one another:
H;
a linear or branched alkyl or alkenyl chain of 1 to 20 carbon atoms, monocyclic, bicyclic or polycyclic of 3 to 20 carbon atoms;
an aryl group of 1 to 20 carbons;
R₃ and R₇ can represent independently of one another:
H;
a linear or branched alkyl or alkenyl chain of 1 to 20 carbon atoms, monocyclic, bicyclic or polycyclic of 3 to 20 carbon atoms;
an aryl group of 1 to 20 carbonss;
R₄ can represent:
H;
a halogen atom
a linear or branched alkyl or alkenyl chain of 1 to 20 carbon atoms, monocyclic, bicyclic or polycyclic of 3 to 20 carbon atoms;
if a represents a single bond, then b represents a single bond and i is equal to 1:
R₁ can represent:
H;
a linear or branched alkyl or alkenyl chain of 1 to 20 carbon atoms, monocyclic, bicyclic or polycyclic of 3 to 20 carbon atoms;
an aryl group of 1 to 20 carbonss;
R₂ can represent:
H;
a linear or branched alkyl or alkenyl chain of 1 to 20 carbon atoms, monocyclic, bicyclic or polycyclic of 3 to 20 carbon atoms;
an aryl group of 1 to 20 carbons;
R₃ can represent:
H;
a linear or branched alkyl or alkenyl chain of 1 to 20 carbon atoms, monocyclic, bicyclic or polycyclic of 3 to 20 carbon atoms;
an aryl group of 1 to 20 carbons;
R₅ and R₇ represent nothing
R₆ represents H
R₄ is as defined above
if a represents a double bond, then b represents a single bond and i is 1:
R₁, R₅, R₆ and R₇ represent nothing;
R₂, R₃ and R₄ are as defined above.

23. Method according to claim 22, said compound being of formula (III) 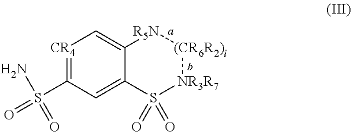
in which:
a can represent:
nothing;
a single bond;
a double bond.
b represents:
nothing if a represents nothing
a single bond if a is either a single bond or a double bond
the value of i being:
zero if a represents nothing,
equal to 1 if a is a single or a double bond;
if a represents nothing, then b represents nothing and i is 0:
R₃ and R₇ can represent independently of one another:
H;
a linear alkyl chain of 1 carbon atom;
R₅ represents H₂;
R₄ can represent:
H;
a halogen atom
a linear alkyl chain of 1 carbon atom;
if a represents a single bond, then b represents a single bond and i is equal to 1:
R₂ can represent:
H;
a linear or branched alkyl or alkenyl chain of 1 to 10 carbon atoms, monocyclic, bicyclic or polycyclic of 3 to 20 carbon atoms;
an aryl group of 1 to 20 carbons;
R₃ can represent:
H;
a linear alkyl chain of 1 carbon atom;
R₅ and R₆ represent H;
R₇ represents nothing;
R₄ is as defined above
if a represents a double bond, then b represents a single bond and i is equal to 1:
R₅, R₆ and R₇ represent nothing
R₂, R₃ and R₄ are as defined above.

24. Method according to claim 22, said compound being of formula (IV) 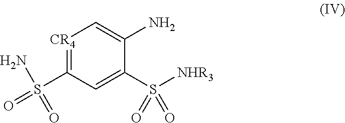
in which:
R₃ can represent:
H;
a linear alkyl chain of 1 carbon atom;
R₄ can represent:
H;
a halogen atom
a linear alkyl chain of 1 carbon atom;
or a compound of formula (IVa).

25. Method according to claim 22, said compound being of formula (V) 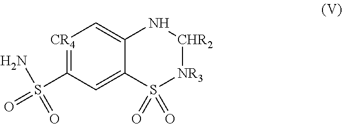
in which:
R₂ can represent:
H;
a linear or branched alkyl or alkenyl chain of 1 to 10 carbon atoms, monocyclic, bicyclic or polycyclic of 3 to 20 carbon atoms;
an aryl group of 1 to 20 carbons;
R₃ can represent:
H;
a linear alkyl chain of 1 carbon atom;
R₄ can represent:
H;
a halogen atom
a linear alkyl chain of 1 carbon atom.

26. Method according to claim 22, said compound being of formula (VI) 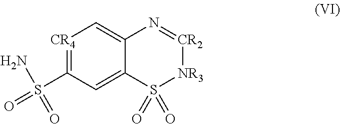
in which:
R₂ can represent:
H;
a linear or branched alkyl or alkenyl chain of 1 to 10 carbon atoms;
an aryl group of 1 to 20 carbons;
R₃ can represent:
H;
a linear alkyl chain of 1 carbon atom;
R₄ can represent:
H;
a halogen atom
a linear alkyl chain of 1 carbon atom.

27. Method, according to claim 22, said compound being althiazide of formula (I) in racemic form:

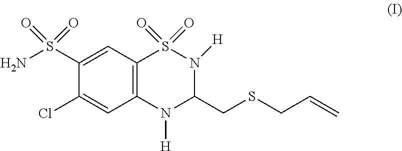 [SMILES: C=CCSCC1Nc2cc(Cl)c(S(N)(=O)=O)cc2S(=O)(=O)N1]

28. Method according to claim 22, said compound being althiazide of formula (IA) and being in pure form or with a chiral purity greater than 99%:

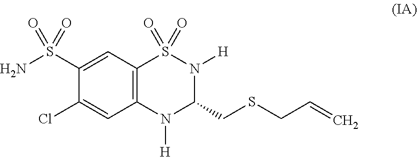 [SMILES: C=CCSC[C@@H]1Nc2cc(Cl)c(S(N)(=O)=O)cc2S(=O)(=O)N1]

29. Method according to claim 22, said compound being althiazide of formula (IB) and being in pure form or with a chiral purity greater than 99%:

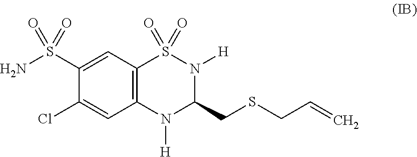 [SMILES: C=CCSC[C@H]1Nc2cc(Cl)c(S(N)(=O)=O)cc2S(=O)(=O)N1]

30. Method according to claim 22, said compound being of formula:

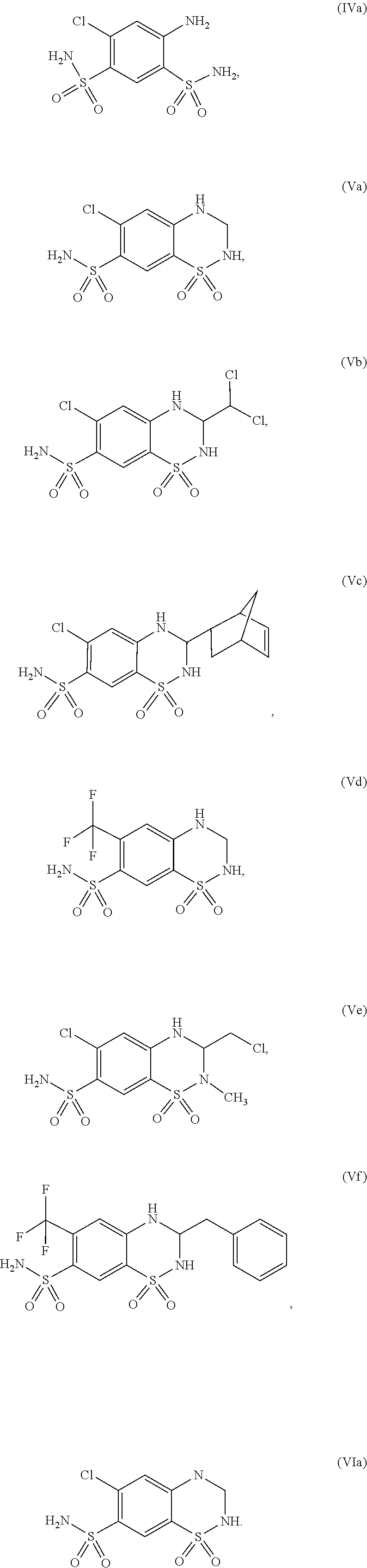

31. Method according to claim 22, wherein the treatment of cancer is the treatment of primary cancer tumors.

32. Method according claim 22, wherein the cancer is in the pre-metastatic state.

33. Method according to claim 22, wherein the compound of formula (II) is an anti-cancer agent or potentiator of an anti-cancer agent, or as an anti-metastatic agent.

34. Method according to claim 22, wherein the compound of formula (II) is in combination with another anti-cancer agent.

35. Method according to claim 22, wherein the compound of formula (II) is an anti-cancer agent or potentiator of an anti-cancer agent, or as an anti-metastatic agent, wherein the compound of formula (II) is in combination with another anti-cancer agent, said other anti-cancer agent being a radiation or a chemical agent, or a chemotherapy agent, a radiotherapy agent, a radio-pharmaceutical or an anti-angiogenic agent.

36. Method according to claim 22, wherein the compound of formula (II) is an anti-cancer agent or potentiator of an anti-cancer agent, or as an anti-metastatic agent, wherein the compound of formula (II) is in combination with another anti-cancer agent, said other anti-cancer agent
being chosen from
alkylating agents such as alkylsulfonates, or busulfan, dacarbazine, procarbazine, cloretazine, nitrogen mustards such as chlormethine, melphalan, chlorambucil, cyclophosphamide, ifosfamide, nitrosoureas such as carmustine, lomustine, semustine, streptozocin, altretamine, fotemustine;
antineoplastic alkaloids such as vincristine, vinblastine, vinorelbine, vindesine;
taxanes such as paclitaxel or taxotere;
antineoplastic antibiotics such as actinomycin, bleomycin;
intercalating agents such as mitoxantrone, etoposide, bleomycin, actinomycin D, amsacrine, alliptinium;
antineoplastic antimetabolites: folate antagonists, methotrexate; inhibitors of purine synthesis; purine analogues such as mercaptopurine, 6-thioguanine; inhibitors of pyrimidine synthesis, aromatase inhibitors, capecitabine, pyrimidine analogs such as fluorouracil, gemcitabine, cytarabine and cytosine arabinoside; brequinar, nelarabine;
group I and II topoisomerase inhibitors such as irinotecan, exatecan, topotecan, teniposide, camptothecin or etoposide;
anticancer hormone agonists and antagonists including tamoxifen;
kinase inhibitors, such as imatinib, nilotinib and dasatinib, midaustorin, sorafenib, lestaurtinib, tandutinib, sirolimus, everolimus or tensirolimus;
growth factor inhibitors;
anti-inflammatories such as pentosan polysulfate, corticosteroids, prednisone, dexamethasone;
ceplene (histamine dihydrochloride);
antracyclines such as daunorubicin, epirubicin, pirarubicin, idarubicin, zorubicin, aclarubicin, annamycin, doxorubicin, mitomycin and methramycin;
anticancer metal complexes, platinum derivatives such as cisplatin, carboplatin, oxaliplatin, satraplatin;
alpha interferon;
triphenylthiophosphoramide;
antiangiogenic agents;
thalidomide;
inhibitors of farnesyl-tranferase such as tipifarnib;
inhibitors of DNA methyltransferase such as MG98;
immunotherapy adjuvants such as gemtuzumab ozogamicin, HuM 195;
biotherapeutic agents such as CT388-I L3;
antisense such as GTI-2040;
vaccines.

37. Method according to claim 22, said compound being formulated to be administered to humans or animals at a dosage from 0.016 mg/kg to 16 mg/kg in admixture with pharmaceutically acceptable excipients.

38. Method according to claim 22, wherein the compound of formula (II) is an anti-cancer agent or potentiator of an anti-cancer agent, said compound being used by oral, parenteral, inhalation spray, nasal, vaginal, rectal, sub-lingual or local administration.

39. Method according to claim 22, wherein the cancer is:
head and neck cancer
lung cancer;
digestive cancer;
gynecological cancer;
a urogenital cancer;
ENT cancer;
breast cancer
cancer of the small intestine;
colon cancer;
liver cancer;
cancer of the bile ducts;
gall bladder cancer;
pancreatic cancer;
kidney cancer;
cancers of the endocrine glands including thyroid cancer, pituitary gland, adrenal gland;
skin cancers including hemangiomas, melanomas, sarcomas, including Kaposi's sarcoma;
tumors of the brain, nerves, eyes, meninges, including astrocytomas, gliomas, glioblastomas, retinoblastomas, neuromas, neuroblastomas, schwannomas, meningiomas;
hematopoietic malignancies; leukemias, (Acute Lymphocytic Leukemia (ALL), Acute Myeloid Leukemia (AML), Chronic Myeloid Leukemia (CML), Chronic lymphocytic leukemia (CLL)) Chloromas, Plasmacytomas, T- or B-cell leukemias, Non-Hodgkin's or Hodgkin's lymphomas, Myeloma, various hematological malignancies;
any form of cancer at high risk metastatic.

40. Method according to claim 22, wherein the compound of formula (II) is in combination with another anti-cancer agent, said other anti-cancer agent being a radiation, or a radiotherapy agent, said cancer being
head cancer
a cancer of the neck;
lung cancer;
a digestive cancer;
gynecological cancer;
urogenital cancer;
ENT cancer;
breast cancer
cancer of the small intestine;
colon cancer;
liver cancer;
bile duct cancer;
gall bladder cancer;
pancreatic cancer;
kidney cancer;
cancers of the endocrine glands including thyroid, pituitary, adrenal gland cancer;
skin cancers including hemangiomas, melanomas, sarcomas, including Kaposi's sarcoma;
tumors of the brain, nerves, eyes, meninges, including astrocytomas, gliomas, glioblastomas, retinoblastomas, neuromas, neuroblastomas, schwannomas, meningiomas; hematopoietic malignancies; leukemia, (Acute Lymphocytic Leukemia (ALL), Acute Myeloid Leukemia (AML), Chronic Myeloid Leukemia (CML), Chronic Lymphocytic Leukemia (CLL)) Chloromas, Plasmacytomas, T- or B-cell leukemias, Non-Hodgkin's or Hodgkin's lymphomas, Myeloma, various hematological malignancies;
any form of cancer at high risk metastatic.

41. Pharmaceutical composition containing a compound of formula (IA) or (IB), in combination with a pharmacologically acceptable carrier,
said compound formula (IA) or (IB) being in pure form or with a chiral purity greater than 99% 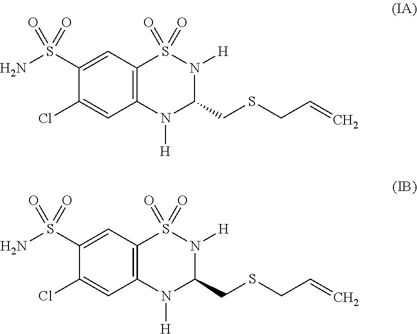 [SMILES: C=CCSC[C@@H]1Nc2cc(Cl)c(S(N)(=O)=O)cc2S(=O)(=O)N1 ; C=CCSC[C@H]1Nc2cc(Cl)c(S(N)(=O)=O)cc2S(=O)(=O)N1]
